(19) United States
(12) Patent Application Publication
(10) Pub. No.: US 2015/0045909 A1
(43) Pub. Date: Feb. 12, 2015

(54) HIGHLY POROUS POLYVINYL HYDROGELS FOR CARTILAGE RESURFACING

(71) Applicants: Orhun K. Muratoglu, Cambridge, MA (US); Hatice Bodugoz-Senturk, Melrose, MA (US)

(72) Inventors: Orhun K. Muratoglu, Cambridge, MA (US); Hatice Bodugoz-Senturk, Melrose, MA (US)

(21) Appl. No.: 14/001,693

(22) PCT Filed: Feb. 22, 2012
(86) PCT No.: PCT/US2012/026074

§ 371 Date: Nov. 5, 2013

Related U.S. Application Data
(60) Provisional application No. 61/447,250, filed on Feb. 28, 2011.

(51) Int. Cl. A61L 27/56; C08J 9/228; A61L 27/52; A61F 2/30; A61L 27/26

(52) U.S. Cl. CPC A61L 27/56; A61F 2/30756; A61L 27/26; A61L 27/52; C08J 9/228; A61L 2430/06
USPC 623/23.72; 523/113; 521/70; 264/41

(57) ABSTRACT

A method of making a creep resistant, highly lubricious, tough hydrogel includes the steps of preparing a first solution including polyacrylamide-co-acrylic acid and another polymer, such as polyvinyl alcohol), and introducing a second solution a gellant into the first solution to form the hydrogel. The first solution can be heated to a first temperature above room temperature, and the combination of the first solution and the second solution can be cooled to a second temperature at or below room temperature. The hydrogel can be used for cartilage repair or in an interpositional device that requires mechanical integrity, high water content, and excellent lubricity in order to fully function under the high stress environment in the joint space and withstand high loads of human joints.

### CROSS-REFERENCES TO RELATED APPLICATIONS

This application claims priority from U.S. Patent Application No. 61/447,250 filed Feb. 28, 2011.

### STATEMENT REGARDING FEDERALLY SPONSORED RESEARCH

Not applicable.

### BACKGROUND OF THE INVENTION

1. Field of the Invention

The invention relates to creep resistant, highly lubricious, tough, and ionic hydrogels, creep resistant, highly lubricious, tough, and ionic hydrogel-containing compositions, and methods of making fabricated ionic hydrogels and ionic hydrogel-containing compositions. The invention also relates to methods of making and using fabricated creep resistant, highly lubricious, tough, and ionic hydrogels including polyvinyl alcohol-polyacrylamide-co-acrylic acid copolymer hydrogels, and creep resistant, highly lubricious, tough, and ionic hydrogel-containing compositions for cartilage repair or as interpositional devices that require mechanical integrity, high water content, and excellent lubricity in order to fully function under the high stress environment in the joint space and withstand high loads of human joints.

2. Description of the Related Art

Osteochondral defect repair in human joints has been a challenging task due to the poor self-healing nature of articular cartilage. The use of non-degradable hydrogel based synthetic materials to repair early cartilage defects has been suggested and explored in animal models. However, defective cartilaginous tissue in the knee, hip, spine, and ear continues to be a major clinical problem. To date, there are no successful strategies for repairing or regenerating cartilaginous tissues with successful long-term outcomes.

Current tissue engineering technology employs degradable polymeric scaffolds to deliver cells in situ. However, degradable scaffolds suffer from weak constructs and incomplete matrix production, and this approach has been met with physiological and technical challenges. The tissue lacks the vascularity needed to regenerate, and the technology lacks strategies that maintain regenerated tissue over the long-term.

Biocompatible hydrogels for cartilage repair or as interpositional devices require mechanical integrity, high water content, and excellent lubricity to fully function under the high stress environment in the human joint spaces. Hydrogels are good candidates for such purposes, but currently available hydrogels may not provide sufficient mechanical strength, creep resistance, and lubricity compatible to that of natural articular cartilage. Most hydrogels systems available for articular cartilage repair or replacement applications do not have required mechanical strength to withstand the high loads of the human joint.

What is needed therefore is a polymeric scaffold having open, interconnected pores and/or continuous channels large enough to allow infiltration of cells and creation of extracellular matrix. A synthetic scaffold infiltrated by living tissue could increase tissue integration in cartilage repair by preserving cells in their natural mechanical environment and enabling biological stimulation thereby allowing extracellular matrix generation. Cartilage replacement could delay the cascade of degeneration and avoid invasive surgical treatments. Also, there remains a need for a creep resistant, highly lubricious, and tough cartilage-like hydrogel composition having ionic moieties and increased the ability to hold water and mechanical strength.

### SUMMARY OF THE INVENTION

Our research focus was to develop a macro porous hydrogel based on polyvinyl alcohol (PVA) or its copolymers, such as polyethylene-co-vinyl alcohol (EVAL), or its blends with other polymers such as polyacrylamide, polyacrylic acid, and/or high molecular weight polyacrylamide-co-acrylic acid copolymers (PAAm-co-AAc) to obtain open, interconnected pores and/or continuous channels large enough to allow infiltration of cells and creation of extracellular matrix. Such a hydrogel with a biological tissue embedded in its channels can be a hybrid device—hybrid in the sense that it will comprise a synthetic scaffold infiltrated by living tissue. Such a device can increase tissue integration in cartilage repair of early arthritic, injured, and diseased human joints to delay degeneration, and in turn, more invasive surgical treatments. It can also be used in tissue augmentation in fields such as plastic reconstructive surgery, urinary tract incontinence, gastro esophageal reflux disease (GERD), etc.

Polyvinyl alcohol (PVA) is one of the most studied polymers for this application due to its viscoelastic nature, high water content, biocompatibility, tailorable mechanical strength, and wide processing window. The strength of PVA-based hydrogels is largely due to their ability to form a semi-crystalline structure through hydrogen bonding of the hydroxyl side groups. However, integration of PVA based hydrogels with the surrounding tissue remains an unsolved problem. We have discovered a method of processing PVA that results in an interconnected, open pore structure to grow any tissue within these pores, including cartilaginous tissue. This novel hydrogel will preserve cells in their natural mechanical environment and enable biological stimulation, therefore allowing extracellular matrix generation.

When prepared by a theta-gel method, PVA hydrogels exhibit porous semi-crystalline gel networks (see U.S. Patent Application Publication No. 2004/0092653, and Bodugoz-Senturk et al., Biomaterials 29 (2) 141-149, 2008). In the theta-gel method, addition of a gelling agent such as low molecular weight poly(ethylene glycol) (PEG) into an aqueous PVA solution reduces the quality of the solvent with decreasing temperature, forcing the PVA to phase separate and crystallize, thus forming a physically crosslinked porous hydrogel network (see FIG. 1). If a solvent is precisely poor enough to cancel the effects of excluded volume expansion, the theta condition is satisfied. For a given polymer-solvent pair, the theta condition is satisfied at a certain temperature, called the theta temperature. A solvent at this temperature is called a theta solvent.

We have discovered that one can alter the pore morphology of the gel network by changing the molecular weight distribution and concentration of PVA and the gelling agent. Adding ionic polyacrylamide-co-acrylic acid copolymer (PAAm-co-AAc) or non-ionic polyacrylamide (PAAm) to a non-ionic PVA solution in a water/gellant mixture resulted in larger pores. We also found that the pores were interconnected and open to the surface, almost like a sponge. Having open interconnected pores or channels allows placement of various cells within these channels and allows nutrient transport to the cells during in vitro culturing and/or in vivo surface after implantation at the site of interest in the human or animal body. The size and distribution of the pores of the gel network are affected by concentration, molecular weight, and the rate of the phase separation.

Another use of this technology is the fixation of non-cellular based implants, such as an osteochondral plugs made out of a hydrogel. A porous PVA-based hydrogel of the current invention can be used to adhere a hydrogel implant to the surrounding tissue. A gradient hydrogel with continuously open and interconnected pore structure on one side and on the opposite side without a continuously open interconnected pore structure can be used for this purpose (see FIG. 2).

In most embodiments, the porosity and the average pore size of PVA-PEG hydrogels were increased by using two or more gelling agents at the same time. The gelation kinetics were altered to control the pore structure by changing the molecular weight and the combination of gelling agents.

In certain embodiments, an ionic or non-ionic component, such as PAAm-co-AAc (ionic) or PAAm (non-ionic), respectively, was added to alter phase separation kinetics. This allowed controlling of the pore structure of the hydrogels.

In some embodiments, the concentration as well as the molecular weight of the host PVA polymer is increased to increase the mechanical strength while keeping the equilibrium water content high.

In certain embodiments, the porous structure and the strength of the PVA hydrogels are altered by dehydration in vacuum, in inert solution, in PEG, in alcohol, and/or in acetone, followed by rehydration cycles in deionized (DI) water or saline.

In some embodiments, high temperature annealing is used to increase the strength of the porous PVA hydrogels subsequent to a dehydration step in vacuum, in inert solution, in PEG, in alcohol, and/or in acetone followed by rehydration cycles in DI water or saline.

In another embodiment, a hybrid (gradient) hydrogel is prepared with a high strength porous component in the bottom layer and a softer porous component on the top layer. The higher strength component is intended to mimic the bone and the softer component is intended to mimic the cartilage layer. While the top layer of such an implant was designed to enhance the cell growth for cartilage formation, the bottom matrix was designed to serve as a base for bone integration as well as to activate the nutrient flow from the blood stream (see FIG. 3).

These and other features, aspects, and advantages of the present invention will become better understood upon consideration of the following detailed description, drawings and appended claims.

### BRIEF DESCRIPTION OF THE DRAWINGS

FIG. 1 shows polyvinyl alcohol-poly(ethylene glycol) (PVA-PEG) theta gel formation.

FIG. 2 shows a schematic of the interaction between soft tissue and a gradient/hybrid hydrogel implant of one embodiment of the invention.

FIG. 3 shows a schematic of a gradient/hybrid hydrogel implant of one embodiment of the invention.

FIG. 4 shows porous polyvinyl alcohol (PVA) hydrogels of one embodiment of the invention with open and interconnected channels.

FIG. 5 shows the chemical structure of polyethylene glycol) (PEG). The value for n can be 200-2000 or higher. It can also be less than 100 or more than 8000.

FIG. 6 shows the chemical structure of polyvinyl alcohol (PVA). The value for n can be 2000-4000 or higher. It can also be less than 2000 or more than 4000.

FIG. 7 shows the chemical structure of polyethylene-co vinyl alcohol (EVAL).

FIG. 8A shows polyvinyl alcohol-polyacrylamide-co-acrylic acid copolymer-polyethylene glycol) (PVA-[PAAm-co-AAc]-PEG) porous hydrogel preparation.

FIG. 8B shows PVA-PEG porous hydrogel preparation.

FIG. 8C shows PVA-[PAAm]-PEG porous hydrogel preparation.

FIG. 9 shows environmental scanning electron microscope (ESEM) images of (1) unimodal PVA-PEG400: a) “de-PEGed” (DP) non-annealed b) “as-gelled”(AG) annealed, and (2) PVA-PEG400-600 bimodal: c) DP non-annealed d) AG annealed.

FIG. 10 shows ESEM images of PVA-(PAAm-coAAc)-PEG(400-600): a) 9-2-10/15% DP non annealed, b) 13.5-1.5-10/15% DP non-annealed, c) 9-2-10/15% AG annealed, and d) 13.5-1.5-10/15% AG annealed.

FIG. 11 shows ESEM images of PVA-(PAAm-coAAc)-PEG(200-400): a) 8.5-2-6/23% DP non annealed, b) 13-1.5-5.5/22% DP non-annealed, c) 8.5-2-6/23% AG annealed, and d) 13-1.5-5.5/22% AG annealed.

FIG. 12 shows ESEM images of polyvinyl alcohol-polyacrylamide-poly(ethylene glycol) (PVA-[PAAm]-PEG) (400-600): a) 9-2-10/15% DP non annealed, b) 13.5-1.5-10/15% DP non-annealed, c) 9-2-10/15% AG annealed, and d) 13.5-1.5-10/15% AG annealed.

FIG. 13 shows ESEM images of PVA-[PAAm]-PEG(200-400): a) 8.5-2-6/23% DP non-annealed, b) 13-1.5-5.5/22% DP non-annealed, c) 8.5-2-6/23% AG annealed, and d) 13-1.5-5.5/22 AG annealed.

FIG. 14 shows a graph of equilibrium water content of PVA-PEG and PVA-(PAAm-co-AAc)-PEG hydrogels in DP (non-annealed) and AG (annealed) forms. The samples are designated in Tables 1 and 3.

FIG. 15 shows a graph of equilibrium water content of PVA-PEG and PVA-(PAAm)-PEG hydrogels in DP (non-annealed) and AG (annealed) forms. The samples are designated in Tables 1-4.

FIG. 16 shows a graph of total creep strain of PVA-PEG and PVA-(PAAm-co-AAc)-PEG hydrogels in DP (non-annealed) and AG (annealed) forms. The samples are designated in Tables 1 and 3.

FIG. 17 shows a graph of total creep recovery of PVA-PEG and PVA-(PAAm-co-AAc)-PEG hydrogels in DP (non-annealed) and AG (annealed) forms. The samples are designated in Tables 1 and 3.

FIG. 18 shows a graph of total creep strain of PVA-PEG and PVA-[PAAm]-PEG hydrogels in DP (not annealed) and AG (annealed) forms. The samples are designated in Tables 1-4.

FIG. 19 shows a graph of total creep recovery of PVA-PEG and PVA-[PAAm]-PEG hydrogels in DP (not annealed) and AG (annealed) forms. The samples are designated in Tables 1-4.

FIG. 20 shows a graph of tear strength of PVA-PEG and PVA-(PAAm-co-AAc)-PEG hydrogels in DP (non-annealed) and AG (annealed) forms. The samples are designated in Tables 1 and 3.

FIG. 21 shows a graph of tear strength of PVA-PEG and PVA-[PAAm]-PEG hydrogels in DP (not annealed) and AG (annealed) forms. The samples are designated in Tables 1-4.

FIG. 22 shows a graph of relative coefficient of friction of PVA-PEG and PVA-(PAAm-co-AAc)-PEG hydrogels in DP (non-annealed) and AG (annealed) form. The samples are designated in Tables 1 and 3.

FIG. 23 shows a graph of relative coefficient of friction of PVA-PEG and PVA-[PAAm]-PEG hydrogels in DP (non-annealed) and AG (annealed) forms. The samples are designated in Tables 1-4.

FIG. 24 shows the preparation of porous PVA-(PAAm-co-AAc)-PEG and Polyethylene-co-vinyl alcohol (EVAL) hybrid hydrogels.

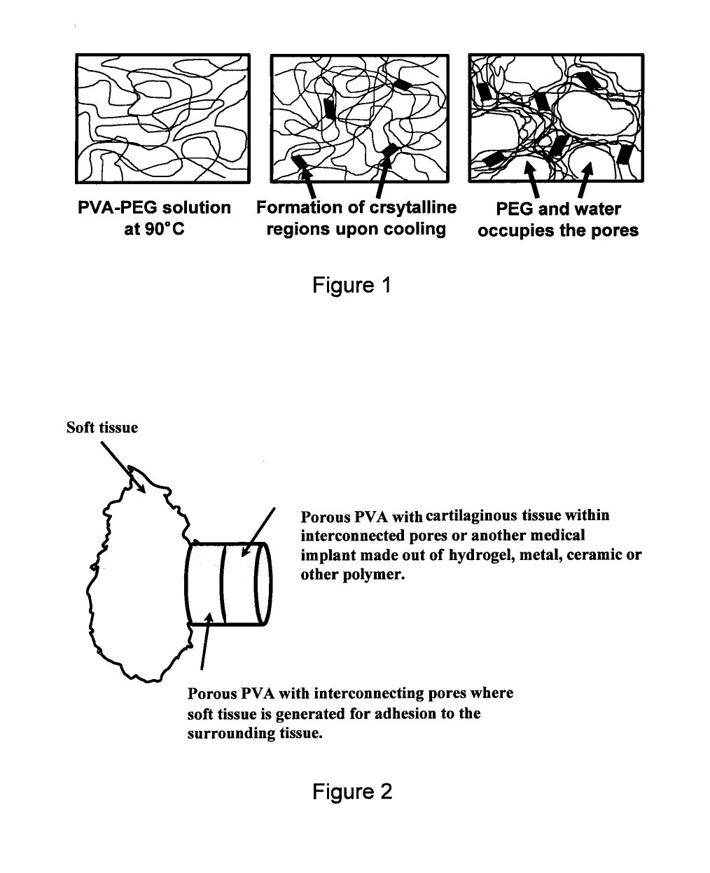

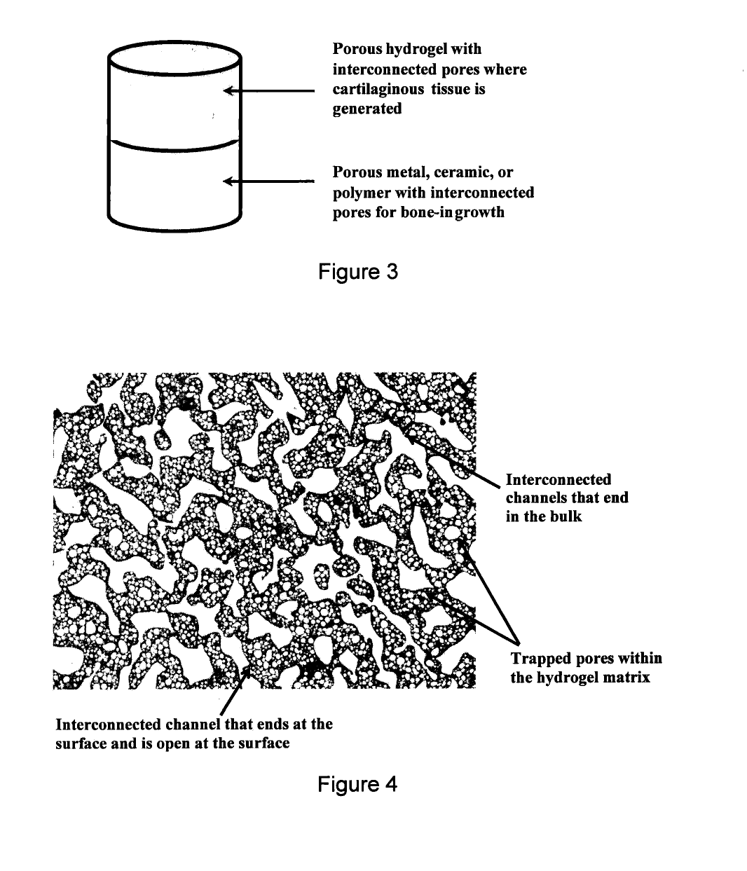

Like reference numerals will be used to refer to like parts from Figure to Figure in the following description of the drawings.

### DETAILED DESCRIPTION OF THE INVENTION

### Definitions

By “porous PVA” is meant a PVA hydrogel containing water and pores. In some embodiments, the pores are trapped within the PVA hydrogel matrix and are occupied by water. In other embodiments, the pores are interconnected and are continuous throughout the PVA hydrogel matrix. In some embodiments, these interconnected open pores are also open at the free surfaces. The pores are typically filled with water or body fluids. In some embodiments, the pores are larger than the cells that are used to grow extracellular matrix in these pores. In other embodiments, the pores are smaller than the cells themselves. Pores, when they are interconnected, are also called channels.

By “pore size” or “channel size” is meant the diameter of the pore or the channel. If it is a trapped pore, the pore shape may be spherical, oblong, or another shape. If non-spherical, an average diameter is used to identify the size of the pore. By channel size is meant the diameter of the channel in its cross-section. The cross-sectional shape of the channel may be a circle, oblong, or another shape. If non-circular, an average diameter is used to describe the channel size.

By “hydrogel” is meant a material that comprises long chain polymers that are physically or chemically crosslinked and coexist with water. Most polymeric materials used in fabricating hydrogels are hydrophilic. They mostly form hydrogen bonds with water and also with each other. The physical crosslinking may be through the formation of hydrogen bonded regions of the polymeric materials. Alternatively, the physical crosslinking may be formed by the formation of small crystalline domains. The water in the hydrogel may exist in a bound form. The bound water is a water molecule that has hydrogen bonded to the polymeric molecules; there is also unbound water in the hydrogel. Some of the unbound water is in the pores of the hydrogel, or it is in the channels of the hydrogel.

In some embodiments, the terms “open pore structure” or “channels” are used interchangeably. They both refer to a porous structure where interconnected continuous pores or channels exist within the hydrogel where these channels end at the surface or in the bulk. Some of the ones that end on the surface are open or exposed at the surface (see FIG. 4).

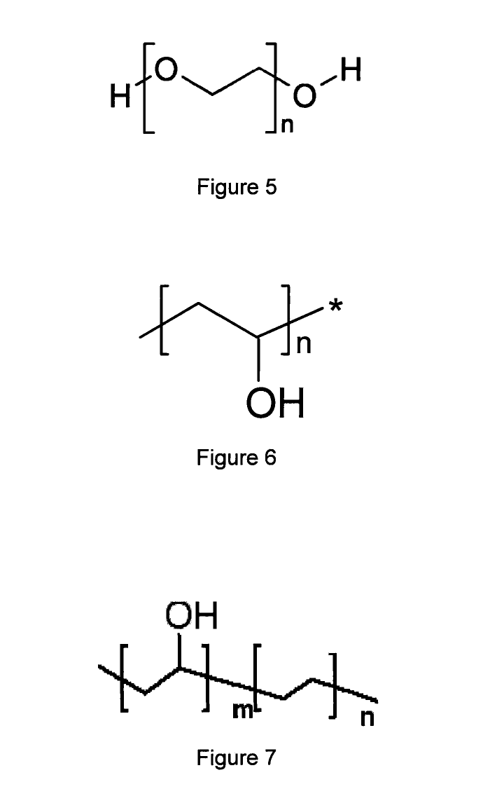

By “PEG” is meant polyethylene glycol. PEG is a molecule with a chemical structure shown in FIG. 5. PEG used in this invention can have a variety of molecular weight distributions. In some embodiments, PEG has a molecular weight of 200 g/mol, or 400 g/mol, or 600 g/mol. The molecular weight of PEG can be less than 200 g/mol, it can be between 200 and 1,000 g/mol, and it can be larger than 1,000 g/mol. In some embodiments, bimodal or multimodal PEG mixtures are used. In the case of a bimodal PEG, two different molecular weight distributions of PEG are utilized as a mixture. For the multimodal applications, more than two PEGs are used all with different molecular weight distributions.

By “PVA” is meant polyvinyl alcohol. PVA is a well-known polymer with a chemical structure shown in FIG. 6. The PVA used in the present invention can have a molecular weight of less than 100,000 g/mol, or it can have a molecular weight of 115,000 g/mol. In some embodiments, the molecular weight is larger than 100,000 g/mol. In some embodiments, PVA with different molecular weight distributions are mixed and used as a mixture in hydrogel making. Some embodiments use bimodal PVA, and others use multimodal PVA.

By “unimodal PEG” is meant a PEG that has a molecular weight distribution that is unimodal. By “bimodal PEG” is meant a PEG that has a molecular weight distribution that is bimodal. By “multimodal PEG” is meant a PEG that has a molecular weight distribution that is multimodal.

By “AG” is meant as-gelled for the hydrogels that are prepared in this invention. The hydrogels are typically prepared by dissolving PVA and PEG and/or a third component in hot water. The solution is cooled down to room temperature (i.e., 20° C.-25° C.). Upon cooling down the hydrogel is formed by physical crosslinking of the PVA molecules or by crystallization of the PVA molecules. This form of the gel is called as-gelled PVA or “AG”. The as-gelled PVA still contains the PEG molecules and/or the third component used in the gelation process. Typically the PEG molecules can be removed from the as-gelled hydrogel by soaking in an appropriate solvent such as DI water. The third component, depending on its diffusability, can also be removed by soaking in an appropriated solvent. However, in this invention AG gels or as-gelled gels are meant to include all of the components within the structure.

By “de-PEGed” is meant removal of PEG and/or the third component as described above from the as-gelled gels. In de-PEGed gels, the removable molecules and/or the removable molecules of the third component are removed by soaking in an appropriate solvent. In some embodiments, the de-PEGed gels are the AG gels that are soaked in DI water for an appropriate amount of time to achieve an equilibrium weight. Typically, the weight of the AG gel changes as the PEG and/or the third component are removed from the hydrogel and water further hydrates the gel. These changes in the weight of the hydrogel reach an equilibrium indicating that the de-PEGing is nearly complete.

By “final concentration” is meant the concentration of various components used in the hydrogel making process. The final concentration is calculated based on the concentration of the components in the solution where all of the components have been added.

By “removable molecules” is meant molecules that exist in a hydrogel and that can be removed by soaking the hydrogel in a solvent such as water, saline, or DI water. In some embodiments, not all molecules are removable as they may be covalently bound to the rest of the hydrogel network or co-crystallized with the hydrogel network.

By “annealing” is meant the heating of the hydrogel. The heating can be carried out in air, in vacuum, or in inert gas. The inert gas can be nitrogen, argon, or helium, or a mixture thereof. The heating rate can vary from 0.01° C./min to 10° C./min. The heating rate can be faster than 10° C./min or slower than 0.01° C./min. In some embodiments, the heating rate will be 0.1° C./min. The annealing temperature may be between 100° C. and 300° C. More preferably, it is 120° C., 130° C., 140° C., 150° C., 160° C., 170° C., 180° C., 190° C., 200° C., 250° C., and 300° C.

By “molecular weight” is meant what is reported by the vendor of a material. For example, the term “weight average molecular weight” (M_{w}) is defined as M_{w}=ΣᵢNᵢMᵢ²/ΣᵢNᵢMᵢ. The term “number average molecular weight” (Mₙ) is defined as Mₙ=ΣᵢNᵢMᵢ/ΣᵢNᵢ. In these calculations, Nᵢ is the number of moles of a polymer of length i, and Mᵢ is the molar mass of the polymer of length i.

By “SRA” is meant soak ramp annealing. In some embodiments, the hydrogels that will be annealed are first subjected to heating below the annealing temperature. In these cases the hydrogels will be soaked at these lower temperatures to obtain some level of dehydration. Subsequently, the hydrogels may be ramped directly to the annealing temperature or they may be ramped to another soak temperature. Multiple soak ramp cycles may be used to further dehydrate the hydrogel. Finally, the hydrogel is heated to the annealing temperature to complete the annealing cycle.

By “EVAL” is meant ethylene vinyl alcohol, a copolymer of ethylene and PVA with a chemical structure as shown in FIG. 7. EVAL is prepared by polymerization of ethylene and vinyl acetate and subsequent hydrolysis of acetate groups. It is commonly defined by the ethylene percentages. EVAL used in the present invention can have a 27% ethylene, or 32%, or 38%, or 44% ethylene.

By “equilibrium water content” is meant the amount of water that a hydrogel can contain at equilibrium. This is measured by weighing the hydrogel after it reaches equilibrium in water, subsequently dehydrating and removing all the removable water from the hydrogel, and weighing it again. The ratio of the difference in weight between the hydrated and dehydrated hydrogel divided by the weight of the hydrated hydrogel is the equilibrium water content.

The present invention provides creep resistant, highly lubricious, tough and ionic hydrogels such as an ionic polyvinyl alcohol-polyacrylamide-co-acrylic acid hydrogel. The hydrogels according to the invention are creep resistant, highly lubricious, tough, cartilage-like, and have increased the ability to hold water. The invention also provides methods of using the fabricated creep resistant lubricious tough ionic hydrogels and creep resistant, highly lubricious, tough and ionic hydrogel-containing compositions for cartilage repair or as interpositional devices that require mechanical integrity, high water content, and excellent lubricity in order to fully function under the high stress environment in the joint space and withstand high loads of human joints.

Hydrogels are sought after for applications in cartilage repair or as interpositional devices. Toughening of a given hydrogel system often results in increased solid content and as a result decreased water content, which may not be desirable for certain applications where lubricity imparted by water in the hydrogel is compromised. One method of toughening hydrogels is through annealing, which increases the creep resistance of polyvinyl alcohol (PVA) but also reduces the equilibrium water content (EWC). We have discovered, among other things, that by adding an ionic hydrophilic compound, such as polyacrylamide-co-acrylic acid (PAAm-co-AAC) into PVA and annealing that mixture, the creep resistance can be increased while maintaining a high level of EWC. PAAm-co-AAC has a hydrophilic nature and high water uptake capability. The ionic hydrogels that are prepared according to the invention disclosed herein are very tough, very creep resistant, very lubricious, and have ionic moieties like the naturally occurring cartilage.

Increasing EWC is beneficial to increase lubrication between the hydrogel and counterface that it will be articulating against in vivo, such as bone, cartilage, metallic or ceramic surfaces, or polymeric materials. The addition of PAAm-co-AAC is not limited to the PVA host polymer; it can be used with other hydrogel systems as well. Copolymers and blends of polyacrylamide-co-acrylic acid can be prepared using PVA as a host polymer, or without PVA. It is generally expected that with addition of ionic groups, a PAAm-co-AAC hydrogel becomes a stimuli response system in which the swelling behavior of hydrogels is affected by environmental conditions such as temperature, ionic strength, and pH of the swelling medium.

The PVA-PAAm-co-AAc hydrogels can be prepared by a number of methods. In one embodiment, a solution of the host PVA hydrogel is mixed with a solution of the PAAm-co-AAC. The mixture is then caused to gel using methods such as theta-gel, radiogel, cryo-gel (freeze/thaw method), or the like.

The theta-gel methodology used in the present invention generates a PVA-PAAm-co-AAc hydrogel through the controlled use of solvents. One method for making a PVA-PAAm-co-AAc hydrogel includes preparing a solution of polyvinyl alcohol and polyacrylamide-co-acrylic acid in a first solvent to form a polymer solution and introducing into the polymer solution a second solvent to cause gelation. The second solvent has a higher Flory interaction parameter at a process temperature than the first solvent. The Flory interaction parameter x is dimensionless and depends on, for example, temperature, concentration and pressure. Solvents can be characterized as having a low x value or solvents having a higher x value. A solvent having a higher x value is characterized as a solvent that causes a gelation process at a temperature. A theta-gel, in accordance with the present invention, is formed by using a second solvent having a Flory interaction parameter that is sufficient to cause gelation.

The mechanism of theta-gel formation includes a phase separation followed by a crystallization mediated by hydrogen bonding in the PVA rich regions of the solution. One desires to control the solvent quality of the system such that the solvent quality is poor enough to accelerate the association rate by promoting the proximity of the PVA chains, while ensuring that the solvent is not so bad that the polymer falls out of solution before crystallization can occur. In general, a PVA-PAAm-co-AAc hydrogel is prepared from an aqueous poly(vinyl alcohol)-polyacrylamide-co-acrylic acid solution that is gelled by contacting with a second solvent having a x value sufficient for gelation. The present method uses a controlled change in solvents differing in solvent quality, conveniently expressed by the Flory interaction parameter to force the PVA to associate. In preferred embodiments, χ of the second solvent must be more positive than the χ of the first solvent (dissolved PVA-PAAm-co-AAc solvent) and is preferably in the range of 0.25 to 2.0. Preferably x of the first solvent is in the range of 0.0 to 0.5. In general, the temperature during processing may vary from just above the freezing point of the PVA-PAAm-co-AAc solution to the melting point of the physical crosslinks formed in the process. One can make a gel with the method of the invention at theta temperature (about 40° C. to 55° C. for the solution described in the method of the invention).

The first solvent is selected from a group of solvents having a low x value that is not sufficient to enable gelation. In example embodiments, the first solvent is selected from the group including, but not limited to, deionized water, dimethyl sulfoxide, a C₁ to C₆ alcohol, and mixtures thereof.

The second solvent, the gellant, is selected from a group of solvents having the property that raises the χ value of the resultant mixture of gellant and PVA-PAAm-co-AAc solution to χ>0.5 at a specified temperature. In example embodiments, the gellant is selected from the group including, but not limited to, polyethylene glycol, alkali salts, glycosaminoglycans, proteoglycans, chondroitin sulfate, starch, dermatan sulfate, keratan sulfate, hyaluronic acid, heparin, heparin sulfate, biglycan, syndecan, keratocan, decorin, aggrecan, perlecan, fibromodulin, versican, neurocan, brevican, a phototriggerable diplasmalogen liposome, amino acids, glycerol, sugars or collagen.

According to one example embodiment, a blend of PVA and PAAm-co-AAC can be mixed with a PEG gellant at a temperature above room temperature so as to cause gelation of the system upon cooling down to room temperature. An aqueous PAAm-co-AAC solution is mixed with an aqueous solution of poly(vinyl alcohol) at an elevated temperature above room temperature (for example, above 30° C., 40° C., 50° C., 55° C., 60° C., 65° C., 70° C., 75° C., 80° C., 85° C., or 90° C.) to form a homogenous PVA-PAAm-co-AAC solution. The PVA to PAAm-co-AAC ratio can be from 0.1:1 to 20:1, or 0.5:1 to 15:1, or 1:1 to 10:1, or 4:1 to 9:1. A PEG solution (i.e., gellant) is then added to the PVA-PAAm-co-AAC solution. The total polymer content in the combined solution can be 1 wt % to 50 wt %, or 3 wt % to 30 wt %, or 5 wt % to 20 wt %, or 10 wt % to 15 wt %. The homogenous PVA-PAAm-co-AAc-PEG solution also can be poured into a mold (optionally pre-heated between 25° C. and 150° C., or between 75° C. and 125° C.) followed by cooling down to a lower temperature (e.g., room temperature) to form a hydrogel.

The gellant can include polymers with different molecular weight distributions. For example, when polyethylene glycol is selected as the gellant, bimodal or multimodal PEG mixtures can be used. In the case of a bimodal PEG, two different molecular weight distributions of PEG are utilized as a gellant mixture. For the multimodal applications, more than two PEGs are used all with different molecular weight distributions. Non-limiting example molecular weights for the PEG include weight average molecular weights (M_{w}) in the range of 100-1000 g/mol, or 100-800 g/mol, or 200-600 g/mol. The level of PEG in a combination of the polymer solution and the gellant solution is preferably in the range of 10 wt % to 50 wt %, or in the range of 15 wt % to 35 wt %, or in the range of 20 wt % to 40 wt %.

Another methodology used in the present invention generates a PVA-PAAm-co-AAc hydrogel by prepolymerizing polyacrylamide-co-acrylic acid in a PVA solution. One can mix a PVA solution with an acrylamide/acrylic acid (AAmAAc) monomer solution containing an initiator and/or catalyst. Example initiators include: thermal initiators such as nitriles (e.g., azobisisobutyronitrile), persulfates (e.g., ammonium persulfate), and peroxides (e.g., benzoyl peroxide); and photoinitiators (e.g., glutaric acid). The AAmAAC in the PVA solution can then be copolymerized and/or cross-linked. The copolymerization of the PAAm-co-AAC in the polymer (such as PVA) solution can be achieved by applying heat or irradiation. A gellant such as polyethylene glycol (PEG) is then added to the solution at a temperature above room temperature. The solution is then cooled to room temperature or below. This results in a porous polyvinyl alcohol-polyacrylamide-co-acrylic acid hydrogel.

According to one aspect of the invention, the PVA-PAAm-co-AAc hydrogel can be post-processed by a variety methods to improve certain properties. Dehydration, annealing by heat, radiation cross-linking and other methods are used to further improve the properties of the hydrogels. The resulting hydrogel is subjected to annealing to further improve its toughness. Preferably, the hydrogel does not lose lubricity upon annealing. Optionally, before annealing the hydrogel is dehydrated using solvent or vacuum dehydration methods. Any residual monomer can be removed by washing the hydrogel with saline, DI water, or alcohol solutions. The unreacted monomer extraction also can be carried out by contacting the hydrogel with a supercritical fluid, such as CO₂ or propane. Another alternative is to crosslink the hydrogels by radiation crosslinking or chemical crosslinking. Optionally, the PVA-PAAm-co-AAc hydrogel is radiation cross-linked before or after monomer removal, before dehydration, after dehydration, and/or after annealing with an optional post-irradiation thermal treatment step.

In another embodiment, a polyvinyl alcohol-polyacrylamide-co-acrylic acid solution is subjected to one or more freeze-thaw cycles (for example, 2, 3, 4, 5, 6, 7, 8, 9, 10 or more cycles) to form the PVA-PAAm-co-AAc hydrogel. According to another embodiment, a PVA-AAmAAC solution with or without a crosslinking agent is first subjected to a freeze-thaw method. This is to cause gelation. Subsequent to gelation PVA-AAmAAC gel is polymerized by applying heat or radiation. In some aspects and embodiments, the polymerized PVA-PAAm-co-AAc is subjected to more freeze-thaw cycle(s) to form a tougher hydrogel.

According to another embodiment, the PVA-PAAm-co-AAc hydrogel is annealed at an elevated temperature either under inert atmosphere in a closed vessel or in a poor solvent such as polyethylene glycol. In some aspects and embodiments, the hydrogel is first dehydrated prior to annealing. Dehydration may be through a number of methods such as, vacuum dehydration, or solvent dehydration (for example, by soaking in polyethylene glycol, isopropanol, ethanol, methanol, or the like).

According to some aspects and embodiments, the PVA-PAAm-co-AAc hydrogel is rehydrated in saturated and dilute NaCl, saturated and dilute KCl, saturated and dilute CaCl₂, or other salt solutions. This is to change the swelling behavior, lubricity and morphology of the gel. By adding salt to the rehydrating solution, the swelling of the gel is decreased, which is then beneficial during the subsequent annealing step.

According to some aspects and embodiments, the PVA-PAAm-co-AAc hydrogel is rehydrated in dilute acid, dilute alkaline and buffer solutions. This is to change the swelling behavior, lubricity and morphology of the gel. By changing the pH of the rehydrating solution, the lubricity and the swelling of the gel are increased which can be beneficial during the subsequent annealing step.

According to another embodiment, a dehydration and annealing step is applied to form a mechanically strong hydrogel. To further increase the mechanical strength, the PVA-PAAm-co-AAc hydrogel is heated. The heating temperature, environment, and duration are varied to tailor the mechanical strength of the PVA-PAAm-co-AAc hydrogel for a specific application. If the heating temperature is above the melting point of the PVA-PAAm-co-AAc hydrogel, then a dehydration step is used to elevate the melting point to above the heating temperatures of the PVA-PAAm-co-AAc hydrogel.

Dehydration can be achieved by a variety of methods, for example, slow heating, vacuum dehydration, and solvent dehydration. For some applications, dehydration followed by rehydration may be sufficient to obtain the desired mechanical properties and annealing may not be necessary in that process.

According to one embodiment of the invention, the mechanical properties of the PVA-PAAm-co-AAc hydrogel can be tailored by changing the ratio of PVA to PAAm-co-AAC and/or by changing the extent of cross-linking induced by the chemical and/or the ionizing radiation routes.

In any of the above embodiments, the final hydrogel can be dehydrated in a solvent or under vacuum and/or subsequently heated prior to final rehydration in water or physiologic saline solution. According to one embodiment, once ionic hydrogels including PAAm-co-AAC are made using any of the above methods described herein, the gels are dehydrated in one or combination of the following environments; in air, vacuum, inert gas, or organic solvents. Dehydration of PAAm-co-AAC containing ionic hydrogels can render PAAm-co-AAC molecules physically trapped inside the PVA gel network by densification, pore collapse, or further polymeric crystallization. Another alternative dehydration method is through soaking the hydrogel in PEG or a PEG solution. The PEG solution could be in any solvent such as water, ethanol, other alcohols, and the like. The PEG solution can vary in concentration between 1% and 100% PEG in the respective solvent. Subsequent to dehydration, the gel can be thermally treated in vacuum, or inert gas at an elevated temperature higher than 100° C., or above or below 160° C., or above 80° C. to about 260° C., for about an hour up to about 20 hours or longer. Such thermal treatments can improve mechanical strength of the gels by further increasing polymer crystallinity. The thermal treatment method described in the polyethylene glycol annealing above also can be done at an elevated pressure rather than the ambient atmosphere.

In some aspects and embodiments, radiation cross-linking in the PVA-PAAm-co-AAc hydrogels processed by methods described here are carried by gamma or e-beam irradiation. The cross-linking increases the wear resistance and creep resistance. The cross-linking can be carried out at any step of the methods described herein.

A hybrid hydrogel can be prepared by sequentially molding different polymers to achieve gradient properties. For example, a hot (for example, about 90° C.) polyethylene-co-vinyl alcohol solution is poured into a container up to a certain thickness and cooled to form a first layer. A hot (for example, about 90° C.) PVA-PAAm-co-AAC-PEG mixture solution is then poured into a container up to a certain thickness to form a second layer. This procedure can be repeated to the desired number of layers or thickness. The gradient properties are thus disposed in a direction perpendicular to the direction of deposit in the mold.

The PVA-PAAm-co-AAc hydrogels and the hybrid hydrogels provided in the present invention can be used in a body to augment or replace any tissue such as cartilage, muscle, breast tissue, nucleus pulpous of the intervertebral disc, other soft tissue, interpositional devices that generally serves as a cushion within a joint, and the like. These PVA-PAAm-co-AAc hydrogels and the hybrid hydrogels provided in the present invention also can be used in the spine for augmenting, replacing the nucleus pulpous, as wound dressing, or as drug delivery vehicles.

PVA-PAAm-co-AAc hydrogels and the hybrid hydrogels of the invention can be used in a variety of fashions in joints in mammals such as human joints. For example, an interpositional device can be manufactured from the PVA-PAAm-co-AAc hydrogels and the hybrid hydrogels, which meet required mechanical strength to withstand high loads of human joints, and can be used in articular cartilage replacement applications. The interpositional devices typically act as a cushion within the joint to minimize the contact of the cartilage surfaces to each other. This is beneficial in patients with arthritic joints. Early arthritic joints with cartilage lesions can be treated with such interpositional devices, which minimize the contact between the damaged cartilage surfaces of the patient. These devices can have a variety of shapes and sizes. For a hydrogel inter positional device to perform in vivo in the long-term, the device first needs to have a high creep resistance. This is to minimize the changes to the shape of the interpositional hydrogel device during in vivo use. PVA-PAAm-co-AAc hydrogels and the hybrid hydrogels of the invention with increased stiffness display increased creep resistance. The hydrogel interpositional device according to the invention also have superior mechanical properties, such as toughness, wear resistance, high creep resistance, high lubricity, cartilage-like ionic moieties, and the like.

Another method for the use of a hydrogel implant is through the filling of a cavity in the joint. The cavity can be an existing one, or one that is prepared by a surgeon. A PVA-PAAm-co-AAc hydrogel or hybrid hydrogel plug can be inserted into the cavity. The hydrogel plug can be of any shape and size; for instance it can be cylindrical in shape. In some aspects and embodiments, the plug can be oversized to be elevated from the surrounding cartilage surface. In other embodiments the plug can be undersized to stay recessed in the cavity. The over-sizing or under-sizing can be such that the plug can stand proud above the surrounding cartilage surface or recessed from the surrounding cartilage surface by about less than 1 millimeter, by about 1 millimeter, by more than about 1 millimeter, by about 2 millimeters, by about 3 millimeters, or by about more than 3 millimeters. In some aspects and embodiments, the hydrogel plug can be slightly dehydrated to shrink its size and to allow an easy placement into the cavity. The hydrogel plug then can be hydrated and swollen in situ to cause a better fit into the cavity. The dehydrated and rehydrated dimensions of the hydrogel plug can be tailored to obtain a good fit, under-sizing, or over-sizing of the plug after rehydration and reswelling. The rehydration in situ can also be used to increase the friction fit between the plug and the cavity. This can be achieved by tailoring the dimensions and the extent of dehydration such that upon rehydration the cross-section of the plug can be larger than the cross-section of the cavity; by for instance about 1 millimeter, less than 1 millimeter, or more than 1 millimeter. In some aspects and embodiments, the cavity can be filled with an injectable hydrogel system including the PVA-PAAm-co-AAc hydrogel.

In another embodiment, the hydrogel-based implant is packaged and sterilized. The packaging can be such that the hydrogel device is immersed in an aqueous solution to prevent dehydration until implantation, such as during sterilization and storage. The aqueous solution can be water, deionized water, saline solution, Ringer's solution, or salinated water. The aqueous solution also can be a solution of polyethylene glycol in water. The solution can be of less than 5 wt % in PEG, about 5 wt %, more than about 5% wt %, about 10% wt %, about 15% wt %, about 20% wt %, about 30% wt %, about 50% wt %, about 90% wt % or about 100% wt %. The hydrogel device also can be sterilized and stored in a non-volatile solvent or non-solvent.

The sterilization of the ionic hydrogel based implant can be carried out through gamma sterilization, heat, gas plasma sterilization, or ethylene oxide sterilization, for example. According to one embodiment, the hydrogel is sterilized by autoclave. The sterilization is carried out at the factory; or alternatively, the implant is shipped to the hospital where it is sterilized by autoclave. Some hospitals are fitted with ethylene oxide sterilization units, which also are used to sterilize the hydrogel implant. In one embodiment, the ionic hydrogel implant is sterilized after packaging.

In some aspects and embodiments, the hydrogel dimensions are large enough so as to allow the machining of a medical device.

In some aspects and embodiments, the hydrogel is shaped into a medical device and subsequently dehydrated. The dehydrated implant is then rehydrated. The initial size and shape of the medical implant is tailored such that the shrinkage caused by the dehydration and the swelling caused by the subsequent rehydration (in most embodiments the dehydration shrinkage is larger than the rehydration swelling) result in the desired implant size and shape that can be used in a human joint.

In certain embodiments, the PVA-PAAm-co-AAc hydrogels and the hybrid hydrogels can be formed or machined into a desired shape to act as medical device, such as a kidney shaped interpositional device for the knee, a cup shaped interpositional device for the hip, a glenoid shaped interpositional device for the shoulder, other shapes for interpositional devices for any human joint. Also the machining of the PVA-PAAm-co-AAc hydrogels and the hybrid hydrogels can result in a cylindrical, cuboid, or other shapes to fill cartilage defects either present in the joint or prepared by the surgeon during the operation.

The PVA-PAAm-co-AAc hydrogel-based and the hybrid hydrogel-based medical device can be an interpositional device such as a unispacer, to act as a free floating articular implant in a human joint, such as the knee joint, the hip joint, the shoulder joint, the elbow joint, and the upper and lower extremity joints.

In some of the aspects and embodiments, the hydrogel is attached to a metal piece. The metal piece can have a porous backside surface that is used for bone-in-growth in the body to fix the hydrogel implant in place. The metal piece attachment to the hydrogel can be achieved by having a porous surface on the substrate where it makes contact with the hydrogel; the porous surface can be infiltrated by the gelling hydrogel solution (for instance a hot PVA-PAAm-co-AAC-PEG mixture in water); when the solution forms a hydrogel, the hydrogel can be interconnected with the metal piece by filling the porous space. In some aspects and embodiments, there can be more than one metal piece attached to the hydrogel for fixation with the hydrogel in the body to multiple locations. In some aspects and embodiments, the hydrogel/metal piece construct can be used during the processing steps described above, such as solvent dehydration, non-solvent dehydration, irradiation, packaging, sterilization, and the like. In some aspects and embodiments, the hydrogel based implants are slightly heated at the surface to partially melt the hydrogel and allow it to reform with more uptake and lubricity.

Thus, the invention provides a method of making a creep resistant, highly lubricious, tough hydrogel. In the method, a first solution including a first polymer and polyacrylamide-co-acrylic acid is prepared, and a second solution including a gellant is introduced into the first solution to form the hydrogel. After introducing the second solution into the first solution, a combination of the first solution and the second solution has a Flory interaction parameter that is sufficient for gelation. In the method, the gellant can be removed from the as-gelled hydrogel, for example by soaking in a solvent such as water.

In one example version of the method, the first polymer is poly(vinyl alcohol). A ratio of the first polymer (e.g., polyvinyl alcohol) to the polyacrylamide-co-acrylic acid in a combination of the first solution and the second solution can be in the range of 0.1:1 to 20:1, or in the range of 0.5:1 to 15:1, or in the range of 1:1 to 10:1, or in the range of 4:1 to 9:1. A total polymer content of the first polymer (e.g., polyvinyl alcohol) and polyacrylamide-co-acrylic acid in a combination of the first solution and the second solution can be in the range of 1 wt % to 50 wt %, or in the range of 3 wt % to 30 wt %, or in the range of 5 wt % to 20 wt %, or in the range of 10 wt % to 15 wt %.

In the method, the first solution can be heated to a first temperature above room temperature, and a combination of the first solution and the second solution can be cooled to a second temperature at or below room temperature. The first temperature can be in the range of 80° C. to 95° C. Alternatively, the first polymer, acrylamide, and acrylic acid can be combined in a mixture, the acrylamide and the acrylic acid can be prepolymerized to polyacrylamide-co-acrylic acid.

In one version of the invention, the gellant is selected from the group consisting of polyethylene glycol, alkali salts, glycosaminoglycans, proteoglycans, chondroitin sulfate, starch, dermatan sulfate, keratan sulfate, hyaluronic acid, heparin, heparin sulfate, biglycan, syndecan, keratocan, decorin, aggrecan, perlecan, fibromodulin, versican, neurocan, brevican, liposomes, amino acids, glycerol, sugars, collagen, and mixtures thereof. Preferably, the gellant is polyethylene glycol. The polyethylene glycol can have a molecular weight distribution with more than one mode. For example, the polyethylene glycol can have a bimodal molecular weight distribution. The polyethylene glycol can have a molecular weight in the range of 100-1000 g/mol, preferably in the range of 100-800 g/mol, and more preferably in the range of 200-600 g/mol. The polyethylene glycol can be present in a combination of the first solution and the second solution in the range of 10 wt % to 50 wt %, or in the range of 15 wt % to 35 wt %, or in the range of 20 wt % to 40 wt %.

In the method, the resulting hydrogel can have an equilibrium water content in the range of 50% to 95%, or in the range of 60% to 95%, or in the range of 70% to 95%. In the method, the resulting hydrogel can have a total creep strain in the range of 50% to 95%, or in the range of 60% to 95%, or in the range of 70% to 95%. In the method, the resulting hydrogel can have a total creep recovery in the range of 10% to 50%, or in the range of 10% to 40%, or in the range of 20% to 40%. In the method, the resulting hydrogel can have a relative coefficient of friction in the range of 0.1 to 1.0, or in the range of 0.1 to 0.8, or in the range of 0.2 to 0.8. In the method, the resulting hydrogel can have a tear strength in the range of 1 to 15 N/m, or in the range of 2 to 15 N/m, or in the range of 2 to 10 N/m.

In the method, a combination of the first solution and the second solution can be placed in a mold. The mold may contain a second hydrogel, and a combination of the first solution and the second solution can be placed into the mold to contact the second hydrogel thereby forming a hybrid hydrogel including the hydrogel and the second hydrogel. Alternatively, a combination of the first solution and the second solution can be placed into a mold to form the hydrogel, and a second hydrogel can be formed in contact with the hydrogel thereby forming a hybrid hydrogel including the hydrogel and the second hydrogel. In one non-limiting example, the second hydrogel is polyethylene-co-vinyl alcohol. The second hydrogel can have a higher hardness than the hydrogel.

In the method, the resulting hydrogel can include channels of interconnected pores. At least some of the channels can be open at a free surface of the hydrogel. The channels can have an average diameter in cross-section between 2 and 100 micrometers. The channels can have an average diameter in cross-section of 100 micrometers or greater. The hydrogel can include pores having an average diameter between 2 and 100 micrometers, or between 2 and 500 micrometers, or between 10 and 300 micrometers, or between 20 and 200 micrometers.

The invention provides another method of making a creep resistant, highly lubricious, tough hydrogel. In this method, an aqueous mixture including a first polymer and polyacrylamide-co-acrylic acid is prepared, and the mixture is subjected to one or more freeze-thaw cycles to form the hydrogel. Preferably, the first polymer is poly(vinyl alcohol).

In one version of the method, the hydrogel can be annealed at a temperature below the melting point of the hydrogel, such as in the range of 80° C. to 300° C., or in the range of 100° C. to 200° C., or in the range of 120° C. to 180° C.

In one version of the method, the hydrogel can be dehydrated under an inert environment or in a dehydrating solvent. For example, the hydrogel can be dehydrated by immersing the hydrogel in a polyethylene glycol solution to allow diffusion of the polyethylene glycol into the hydrogel. After dehydrating, the hydrogel can be annealed at a temperature of about 80° C. to about 200° C. After dehydrating, the hydrogel can be rehydrated by soaking in a saline solution or in water.

In one version of the method, the hydrogel is contacted with an organic solvent, wherein the hydrogel is not soluble in the solvent, and wherein the solvent is at least partially miscible in water. The hydrogel is heated to a temperature below or above the melting point of the hydrogel, and the heated hydrogel is cooled to room temperature. Alternatively, the hydrogel can be air-dried at room temperature after being contacted with an organic solvent. Alternatively, the hydrogel can be subjected to at least one freeze-thaw cycle and allowed to warm-up room temperature after being contacted with an organic solvent.

In one version of the method, the hydrogel is dehydrated by placing the hydrogel in (i) a non-solvent selected from the group consisting of polyethylene glycol, alcohols, acetones, saturated salinated water, vitamin, carboxylic acids, and aqueous solutions of a salt of an alkali metal, or (ii) in a supercritical fluid.

In one version of the method, the hydrogel is dehydrated at a first temperature and then heated in air or in inert gas to an elevated temperature using a heating rate ranging from about 0.01° C./minute to about 10° C./minute.

In one version of the method, the hydrogel is dehydrated, and then rehydrated by placing the dehydrated hydrogel (i) in water, saline solution, Ringer's solution, salinated water, or buffer solution, or (ii) in a humid chamber, or (iii) at room temperature or at an elevated temperature. The hydrogel can be rehydrated to reach an equilibrium. The hydrogel can be rehydrated in water or a salt solution.

Hydrogels made by a method according to the invention can be used in a medical implant. One example implant is an interpositional device wherein the interpositional device a unispacer, and the unispacer is a free floating articular implant in a human joint such as a knee, a hip, a shoulder, an elbow, or an upper or an extremity joint. The medical implant can be packaged and sterilized. The medical implant can be sterilized by ionizing radiation. The medical implant can be sterilized by gamma or E-beam radiation at a dose between about 25 kGy to about 200 kGy.

Another example medical implant includes a first layer comprising a first hydrogel made by a method according to the invention, and a second layer attached to the first layer, wherein the second layer comprises a second material selected from metallic materials, ceramic materials and polymeric materials. The second layer can comprise a second hydrogel made by a method according to the invention, and the first hydrogel and the second hydrogel can have different properties. For example, the first hydrogel and the second hydrogel can have different pore structures. The first hydrogel can have a continuously open and interconnected pore structure, and the second hydrogel does not have a continuously open interconnected pore structure. The second material can have a higher hardness than the first hydrogel. In one form, the second material is polyethylene-co-vinyl alcohol.

The first hydrogel and/or the second hydrogel can include pores at least partially filled with a bioactive agent selected from the group consisting of enzymes, organic catalysts, ribozymes, organometallics, proteins, glycoproteins, peptides, polyamino acids, antibodies, nucleic acids, steroidal molecules, antibiotics, antimycotics, cytokines, cells, growth factors, carbohydrates, oleophobics, lipids, extracellular matrix and/or its individual components, pharmaceuticals, therapeutics, and mixtures thereof. The first hydrogel and/or the second hydrogel can include pores at least partially filled with a bioactive agent selected from the group consisting of growth factors, chondrocyte precursor cells, mesenchymal stem cells, chondrocytes, and mixtures thereof.

### EXAMPLES

The following Examples have been presented in order to further illustrate the invention and are not intended to limit the invention in any way.

Several different formulations of the porous PVA hydrogels were prepared by varying the processing parameters. These are listed in Table 1. The examples below use one or more of these formulations. Table 1 shows PVA-(PAAm-co-AAc)-PEG, PVA-PEG400 and PEG400/PEG600 hydrogel formulations. All concentrations are weight percent of the final DI water solution after all of the components were added in DI water.

TABLE 1

| | Sample | | | | | |
|---|---|---|---|---|---|---|
| | Desig- | | PAAm- | | | |
| | nation | PVA | co-AAc | PEG | | |
| Sample | Letter | (%) | (%) | 200 | 400 | 600 |
| 9PVA-2PAAm-co- | A | 9 | 2 | — | 10 | 15 |
| AAc-10/15PEG400/600 | | | | | | |
| 8.5PVA-2PAAm-co- | B | 8.5 | 2 | 6 | 23 | |
| AAc-6/23PEG200/400 | | | | | | |
| 13.5-PVA-1.5PAAm- | C | 13.5 | 1.5 | — | 10 | 15 |
| co-AAc-10/15PEG400/ | | | | | | |
| 600 | | | | | | |
| 13PVA-1.5PAAm-co- | D | 13 | 1.5 | 5.5 | 22 | — |
| AAc-5.5/22PEG200/ | | | | | | |
| 400 | | | | | | |
| 11 PVA-25PEG400 | E | 11 | — | — | 25 | — |
| 11 PVA- | F | 11 | — | | 10 | 15 |
| 10/15PEG400/600 | | | | | | |

The total concentration of PVA (%) = w PVA/(wPVA + PAAm-co-AAc + PEGs + Water)
The total concentration of PEG(or PEG mixtures) = w PEG/(w PVA/(wPVA + PAAm-co-AAc + PEGs + Water)

### Example 1

### Preparation of the PVA-(PAAm-co-AAc)-PEG Hydrogels

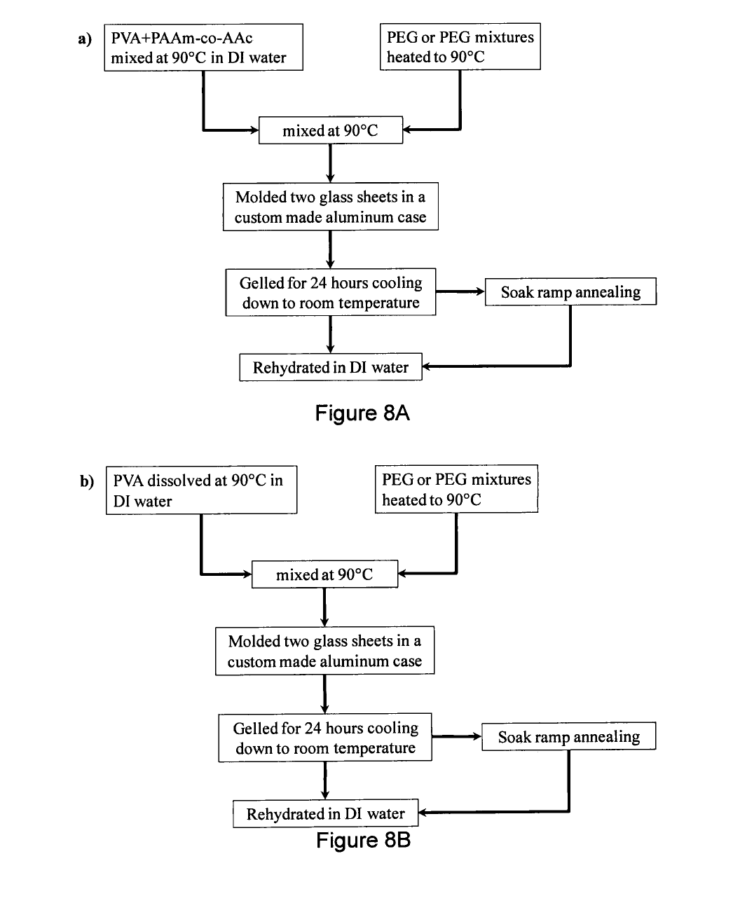

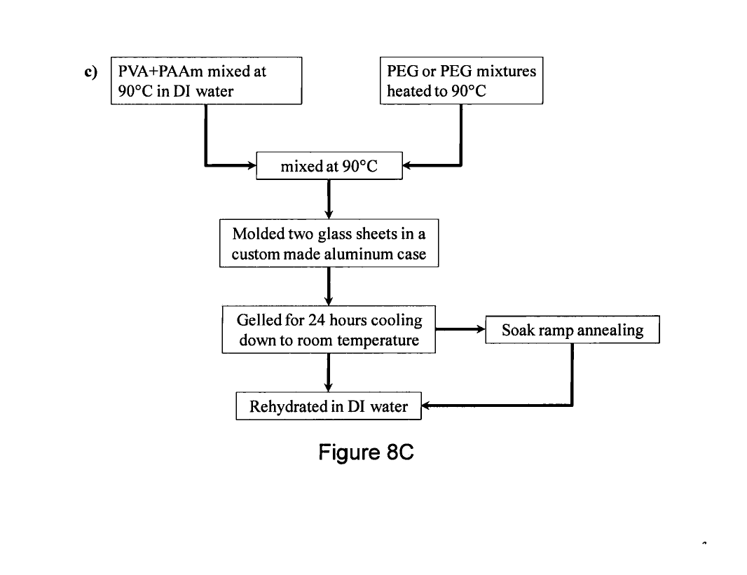

PVA-(PAAm-co-AAc-PEG) theta gels were prepared by dissolving 9% PVA and 2% PAAm-co-AAc in DI water and mixing this solution at 90° C. with a pre-heated mixture of low and high molecular weight PEG. The PEG mixture was bimodal and consisted of PEG400 (M_{w}=400) and PEG600 (M_{w}=600). The PEG400 was 10% in the final solution and the concentration of PEG600 was 15% (Final solution=PVA+PAAm-co-AAc+PEG400+PEG600+DI water). The concentrations are in weight percentages (see sample designation letter A in Table 1). The final solution was poured between two glass sheets in a custom made aluminum case and gelled for 24 hours and cooled down to room temperature (see FIG. 8). After gelation, two groups were prepared: “as-gelled” (AG) and “de-PEGed” (DP). The latter was immersed in DI solution in order to remove PEG and remove unreacted PAAm-co-AAc from the hydrogel on a rotary shaker at room temperature for at least 7 days. Equilibrium was determined by periodically weighing the gels.

Another gel was prepared to investigate the effect of PEG molecular weight (see sample designation letter B in Table 1). 8.5% PVA was dissolved in the presence of 2% PAAm-co-AAc in DI water and mixed with 6% PEG200 and 23% PEG400 mixture at 90° C. as described above. Various other concentrations were prepared following the above described method (Table 1). A 13.5% PVA-1.5% PAAm-co-AAc hydrogel was prepared by using 10% PEG400-15% PEG600 (see sample designation letter C in Table 1), and a 13% PVA-1.5% PAAm-co-AAc hydrogel was prepared by using 22% PEG400-5.5% PEG200 (see sample designation letter D in Table 1).

For further comparison, two PVA-PEG theta gels which did not contain any PAAm-co-AAc polymer were prepared: one unimodal formulation with PEG400 only, and the other with bimodal formulation using PEG400/PEG600. Where PEG400 was 40% of the PEG400/PEG600 mixture and PEG600 was 60%. PVA-PEG400 theta gels were prepared by first dissolving 11% (w/w) PVA (M_{w}=115,000 g/mol) in deionized (DI) water at 90° C. PEG400 at a final concentration of 25% was added to this solution while stirring. The mixture of solutions was poured in a mold and cooled down to room temperature for gelation (sample designation letter E in Table 1).

PVA-PEG400/PEG600 theta-gels were prepared by dissolving 11% (w/w) PVA (M_{w}=115,000 g/mol) in deionized water at 90° C. PEG mixture consisting of 10% PEG400 in the final solution and 15% PEG600 in the final solution was prepared and heated up to 90° C. then added to the PVA solution at 90° C. while stirring (see sample designation letter F in Table 1).

For both PVA-PEG400 and PVA PEG400/PEG600 formulations, two gel groups were prepared. One group was used in their “as-gelled” form (AG); the other group was immersed in DI after gelation for PEG removal and continued to be used in this “dePEGed” form (DP). The latter was immersed in DI solution in order to remove the PEG and unreacted PAAm-co-AAc on a rotary shaker for at least 7 days at room temperature. Equilibrium was determined by periodically weighing the gels.

### Example 2

### Preparation of the PVA-[PAAm]-PEG) Hydrogels

A PVA-PAAm-PEG theta gel was prepared by dissolving 9% PVA and 2% PAAm in DI water and mixing this solution at 90° C. to a mixture of low and high molecular weight PEG mixture (10% PEG400 and 15% PEG600 in the final solution). See sample designation letter G in Table 2.

A PVA-PAAm-PEG theta gel was prepared by dissolving 8.5% PVA and 2% PAAm in DI water and mixing this solution at 90° C. to a mixture of low and high molecular weight PEG mixture (6% PEG200 and 23% PEG400 in the final solution). See sample designation letter H in Table 2.

A PVA-PAAm-PEG theta gel was prepared by dissolving 13.5% PVA and 1.5% PAAm in DI water and mixing this solution at 90° C. to a mixture of low and high molecular weight PEG mixture (10% PEG400-15% PEG600 in the final solution). See sample designation letter I in Table 2.

A PVA-PAAm-PEG theta gel was prepared by dissolving 13.5% PVA and 1.5% PAAm in DI water and mixing this solution at 90° C. to a mixture of low and high molecular weight PEG mixture (5.5% PEG200-22% PEG400 in the final solution). See sample designation letter J in Table 2.

The solutions were poured between two glass sheets in a custom made aluminum case and gelled for 24 hours and cooled down to room temperature. After gelation, two groups were prepared: “as-gelled” (AG) and “de-PEGed” (DP). The latter molded gels were immersed in DI solution in order to remove the removable PEG and removable unreacted PAAm from the hydrogel on a rotary shaker at room temperature for at least 7 days. Equilibrium was determined by periodically weighing the gels.

AG groups of PVA-PEG, PVA-[PAAm-co-AAc]-PEG and PVA-PAAm-PEG hydrogels were dried in a convection oven at 25° C. for 14 hours, ramped to 80° C. in 8 hours, then kept at 80° C. for 20 hours with a subsequent annealing period of 20 hours at 160° C. under argon in a sealed stainless steel vessel (soak ramp annealing). During annealing the sealed vessel self-pressurized as a result of heating. The self-generated pressure was about 5.5 atm. The annealed gels were rehydrated in DI water until equilibrated to remove the PEG and unreacted PAAm-co-AAc or PAAm polymer.

Table 2 shows PVA-[PAAm]-PEG hydrogel formulations.

TABLE 2

| | Sample | | | | | |
|---|---|---|---|---|---|---|
| | Desig- | | | | | |
| | nation | PVA | PAAm | PEG | | |
| Sample | Letter | (%) | (%) | 200 | 400 | 600 |
| 9PVA-2PAAm -10/ | G | 9 | 2 | — | 10 | 15 |
| 15PEG400/600 | | | | | | |
| 8.5PVA-2PAAm -6/ | H | 8.5 | 2 | 6 | 23 | |
| 23PEG200/400 | | | | | | |
| 13.5-PVA-1.5PAAm - | I | 13.5 | 1.5 | — | 10 | 15 |
| 10/15PEG400/600 | | | | | | |
| 13PVA-1.5PAAm - | J | 13 | 1.5 | 5.5 | 22 | — |
| 5.5/22PEG200/400 | | | | | | |

The total concentration of PVA (%) = w PVA/(wPVA + PAAm + PEGs + Water)
The total concentration of PEG(or PEG mixtures) = wPEG/(w PVA/(wPVA + PAAm + PEGs + Water)

### Example 3

### ESEM Imaging

The AG and DP groups of PVA-PEG, PVA-[PAAm-co-AAc]-PEG and PVA-[PAAm]-PEG gels were imaged by using a FEI/Philips XL30 FEG ESEM microscope. All samples for ESEM were cryofractured by immersing in liquid nitrogen and subsequently rehydrating in DI water.

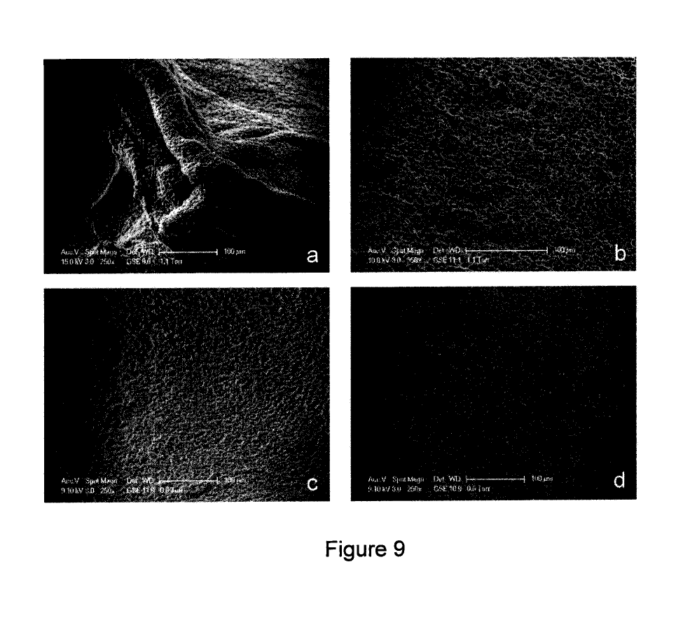

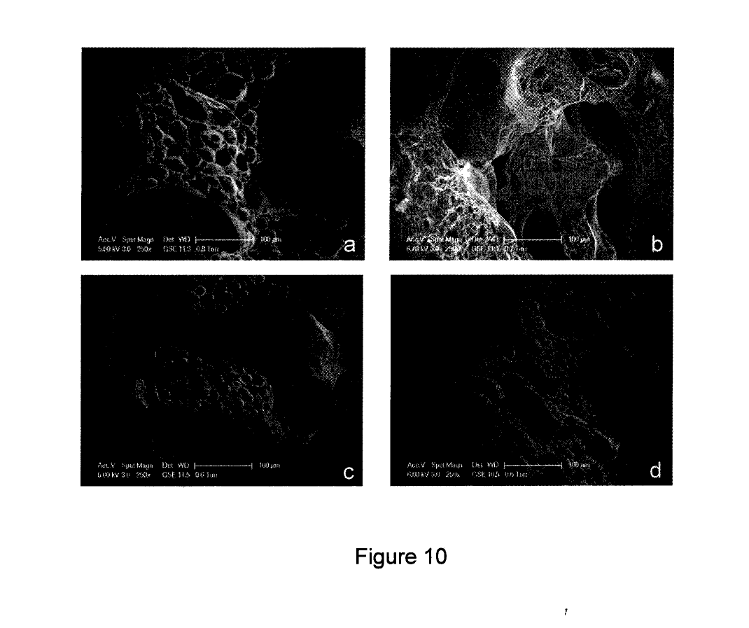

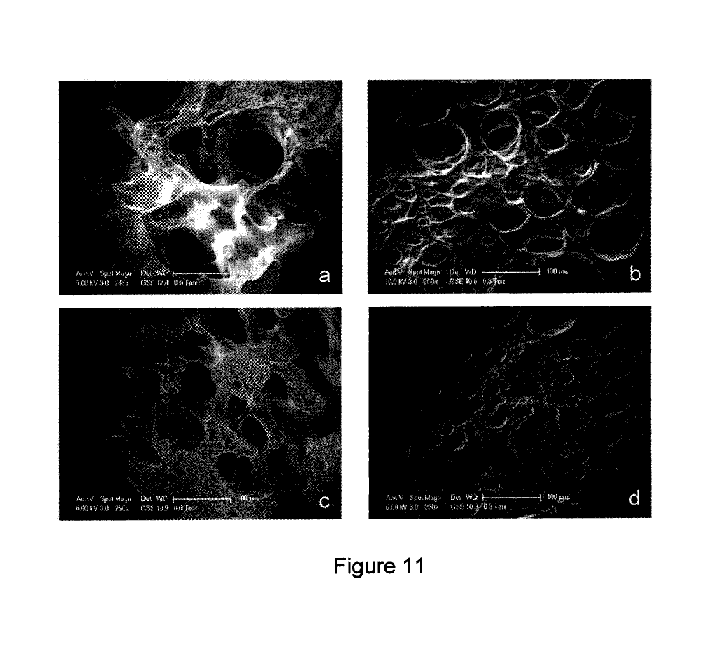

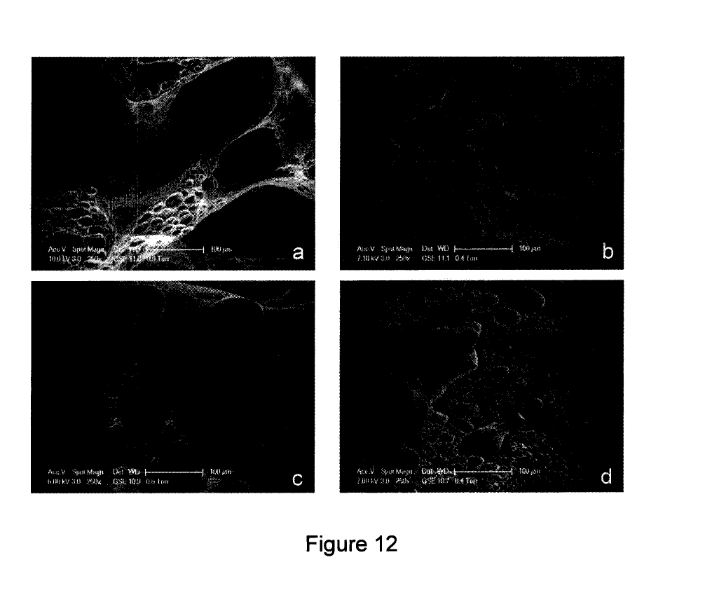

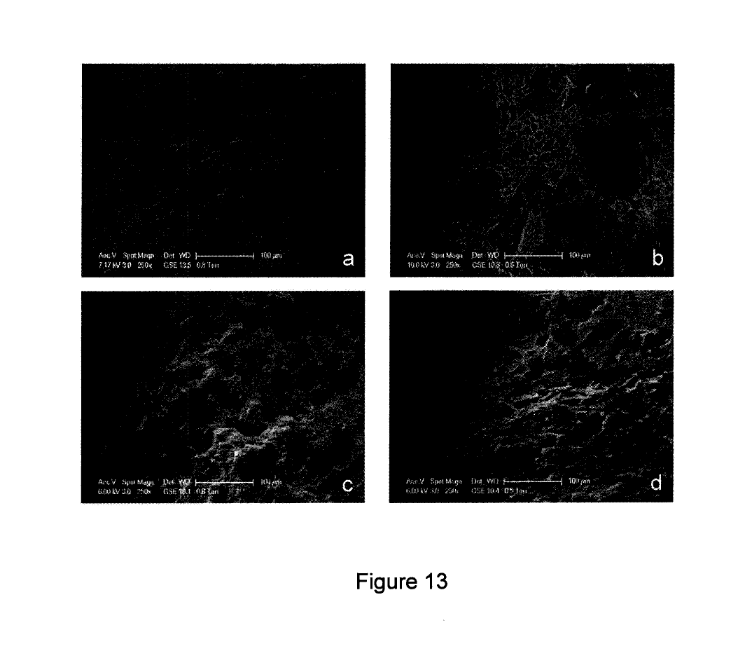

PVA-[PAAm-co-AAc]-PEG and PVA-PAAm-PEG formulations both with high (13% or 13.5% in the final solution) and low PVA (8.5% or 9% in the final solution) content with PEG400-600 and PEG200-400 gellant mixtures showed bigger pores than unimodal or bimodal PVA-PEG formulations (see FIGS. 9-13). Hydrogels with PAAm-co-AAc and PAAm showed pores bigger than 100 μm along with the smaller pores ranging in size between 2 and 100 μm. Overall, ESEM images suggested that the bigger pores were interconnected (see FIGS. 9-12). PVA concentration influenced the pore size and distribution in both PVA-(PAAm-co-AAc)-PEG and PVA-PAAm-PEG hydrogels (see FIGS. 10-13); higher PVA concentration (13% in final solution) resulted in smaller pores compared to their low PVA concentration counterparts (see FIG. 10a-10b, FIG. 11a-11b, FIG. 12a-12b, and FIG. 13a-13b). Annealing did not result in a visible change in the pore size (see FIGS. 10a-10c and 10b-10d, FIGS. 11a-11c and 11b-11d, FIGS. 12a-12c and 12b-12d, and FIGS. 13a-13c and 13b-13d). This was true at both high and low PVA content.

Although the structure appeared to have smaller pores in the PVA-PEG formulation with PEG200/PEG400, compared to PEG400/PEG600, changing the PEG molecular weight distribution in the PEG mixture in PVA-(PAAm-co-AAc)-PEG and PVA-[PAAm]-PEG mixtures did not affect the pore size or the distribution extensively (see FIGS. 10-13). In addition, both PVA-PAAm-PEG200/PEG400 and PVA-PAAm-co-AAc-PEG200-400 hydrogels had smoother surfaces, especially with 13% or 13.5% PVA concentration compared to their PEG400/PEG600 counterpart.

### Example 4

### Equilibrium Water Content

The equilibrium water content (EWC) of both AG-annealed and DP-unannealed groups of PVA-PEG, PVA-[PAAm-co-AAc]-PEG and PVA-[PAAm]-PEG gels was measured using a Thermogravimetric Analyzer (TGA) (Q500, TA Instruments, New Castle, Del., USA). The gels were first immersed in DI water with agitation until equilibrium hydration. Three samples of approximately 20 mg were cut from each equilibrated hydrogel and heated at a rate of 20° C./minute from 25° C. to 200° C. under a nitrogen purge. The weight change of the samples was determined by taking the difference between the initial weight and the equilibrium dried weight. The percent equilibrium water content was determined by dividing the weight change over the initial weight.

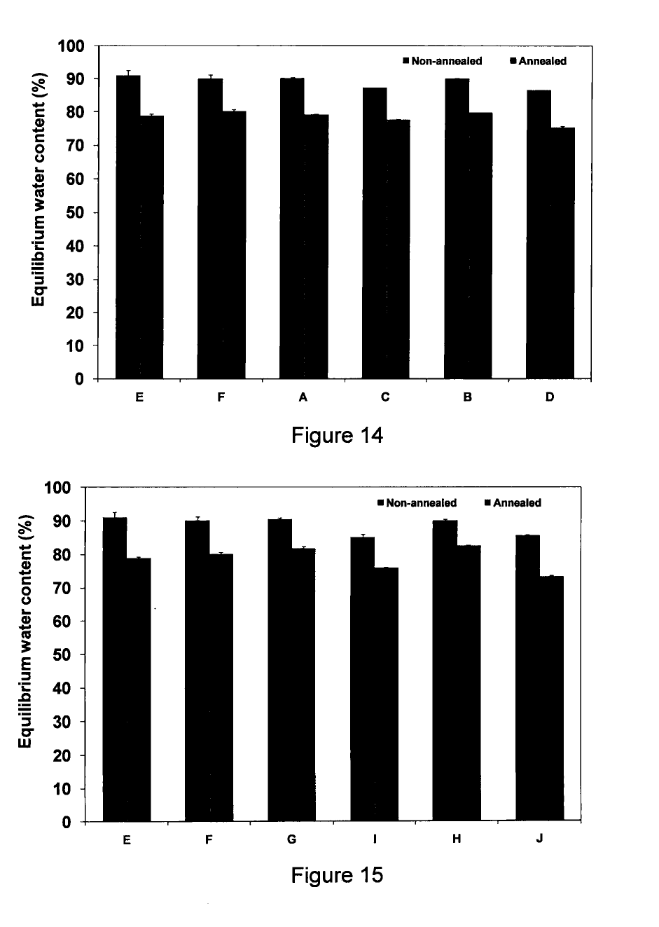

For all types of hydrogels prepared, DP (not annealed) group showed higher EWC compared to their annealed AG (annealed) counterpart. The difference in the EWC of PVA-PEG400, PVA-PEG400-600 formulations with 8.5% or 9% PVA and PVA-(AAm-co-AAc)-PEG and PVA-PAAm-PEG formulation with lower PVA concentration was not significant. However, increasing the PVA concentration to 13% or 13.5% in PVA-(AAm-co-AAc)-PEG and PVA-PAAm-PEG formulations resulted in a decrease in EWC, specifically in unannealed form of these hydrogels (see FIGS. 14-15 and Tables 3-4 below).

Table 3 shows equilibrium water content (EWC), relative coefficient of friction (RCOF) and tear strength (TEAR) of PVA-PEG and PVA-(PAAm-co-AAc)-PEG hydrogels in DP (non-annealed) and AG (annealed) form. The designations A-J are the same as in Tables 1 and 2.

TABLE 3

| | EWC | | TEAR |
|---|---|---|---|
| Sample | (%) | RCOF | (N/m) |
| DePEGed | | | |
| E | 91 ± 2 | 0.3 ± 0.001 | 0.3 ± 0.06 |
| F | 90 ± 1 | 0.3 ± 0.003 | 1 ± 0.4 |
| A | 90 ± 0.3 | 0.3 ± 0.01 | 2 ± 0.1 |
| C | 87 ± 0.2 | 0.4 ± 0.04 | 4 ± 0.4 |
| B | 86 ± 0.4 | 0.6 ± 0.04 | 2 ± 0.1 |
| D | 90 ± 0.5 | 0.3 ± 0.04 | 3 ± 0.1 |
| AG-Annealed | | | |
| E | 79 ± 0.6 | 0.5 ± 0.001 | 9 ± 0.2 |
| F | 80 ± 0.7 | 0.4 ± 0.02 | 9 ± 0.8 |
| A | 79 ± 0.4 | 0.7 ± 0.05 | 7 ± 0.4 |
| C | 78 ± 0.2 | 0.8 ± 0.08 | 10 ± 0.1 |
| B | 80 ± 0.3 | 0.4 ± 0.02 | 4 ± 0.3 |
| D | 75 ± 0.3 | 0.5 ± 0.004 | 11 ± 0.4 |

Table 4 shows equilibrium water content (EWC), relative coefficient of friction (RCOF) and tear strength (TEAR) of PVA-PEG and PVA-[PAAm]-PEG hydrogels in DP (non-annealed) and AG (annealed) form. The designations A-J are the same as in Tables 1 and 2.

TABLE 4

| | EWC | | TEAR |
|---|---|---|---|
| Sample | (%) | RCOF | (N/m) |
| DP-Non-annealed | | | |
| G | 90 ± 0.4 | 0.7 ± 0.07 | 2.5 ± 0.1 |
| I | 85 ± 1 | 0.7 ± 0.06 | 4 ± 0.1 |
| H | 90 ± 0.4 | 0.4 ± 0.06 | 2 ± 0.1 |
| J | 86 ± 0.3 | 0.5 ± 0.06 | 4 ± 0.2 |
| AG-Annealed | | | |
| G | 82 ± 0.6 | 0.3 ± 0.02 | 8 ± 0.7 |
| I | 76 ± 0.2 | 0.3 ± 0.02 | 11 ± 0.4 |
| H | 82 ± 0.3 | 0.2 ± 0.03 | 5 ± 0.4 |
| J | 73 ± 0.6 | 0.3 ± 0.03 | 13 ± 0.7 |

### Example 5

### Creep Test

The creep behavior of AG-annealed and DP-unannealed groups of PVA-PEG, PVA-[PAAm-co-AAc]-PEG and PVA-[PAAm]-PEG gel samples was assessed. Hydrogels were cut into cylindrical disks with a 16 mm diameter trephine mounted on a drill press while submerged in saline at room temperature. The creep test samples were allowed to equilibrate for 24 hours in DI water at 40° C. before testing. The creep experiments were performed on a custom-built mechanical tester in 40° C. DI water. Test samples were placed between the compression plates and loaded under compression to 100 N at a rate of 50 N/min, resulting in an initial compressive stress of about 0.45 MPa. This load was maintained constant for 10 hours. The load was then reduced to a rate of 50 N/min to a recovery load of 10 N. This load was also held constant for 10 hours. Time, displacement, and load were recorded once every two seconds during the loading and unloading cycles. Creep strain was calculated as (1) the elastic creep strain (ES) at the completion of ramp-up to 100N load, (2) the viscoelastic creep strain (VS) after 10 hours of loading, (3) the total creep strain (TCS) after 10 hours of loading, (4) the elastic creep strain recovery (ER) upon unloading from 100N to 10N, (5) the viscoelastic creep strain recovery (VR) after 10 hours of unloading under 10 N, (6) the total strain recovery (TR) after 10 hours of unloading under 10N, and (7) the total strain (permanent deformation) (FS) after 10 hours of loading followed by 10 hours of unloading under 10 N. The total creep strain (TSC) was taken as a representative creep characteristic of each formulation studied.

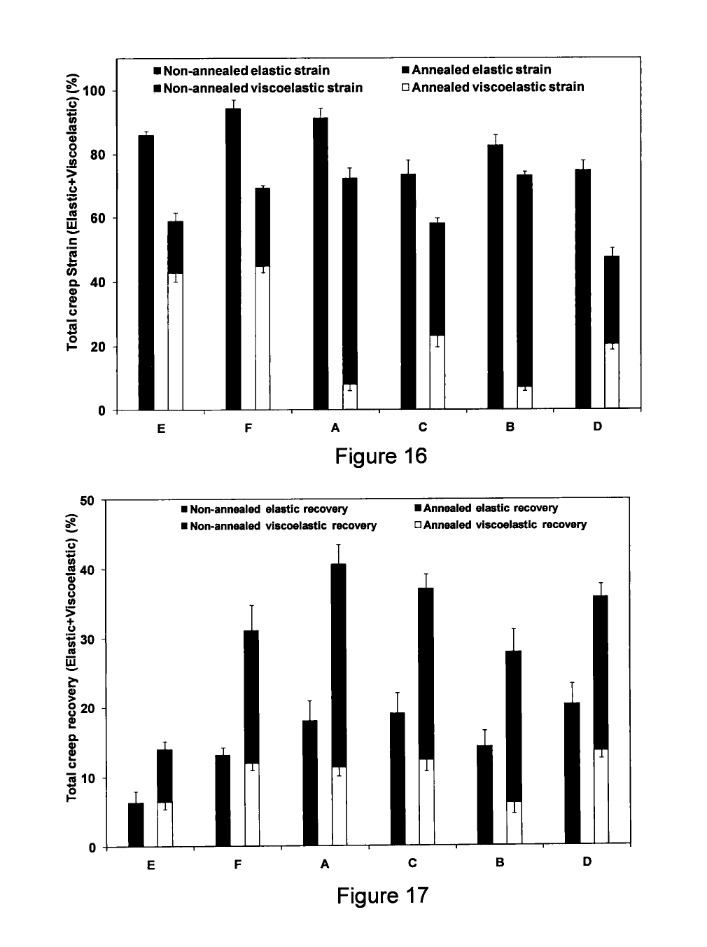

The creep and recovery behavior of the PVA-PEG and PVA-(PAAm-co-AAc)-PEG hydrogels are summarized in FIGS. 16-17 and Table 5 below. Annealing markedly improved the creep resistance of all formulations studied. Increasing PVA content resulted in an increase in the creep resistance of highly porous PVA-(PAAm-co-AAc)-PEG both before and after annealing (see FIG. 16). In their non-annealed form, the initial elastic response of the gels to the 100N load was slightly lower in the high PVA content PVA-(PAAm-co-AAc)-PEG formulation than PVA-PEG formulations with the smaller pore structure. In the annealed form of PVA-[PAAm-co-AAc]-PEG, for hydrogels with 8.5% or 9% and 13% or 13.5% PVA formulations, the elastic component was significantly higher than that measured with PVA-PEG formulations prepared using either unimodal or bimodal PEG. While the viscoelastic strain for the non-annealed form of all hydrogels was similar, highly porous PVA-(PAAm-co-AAc)-PEG hydrogels showed significantly lower viscoelastic strain than PVA-PEG hydrogels in their annealed form. PEG molecular weight did not show a significant effect on the creep behavior of PVA-[PAAm-co-AAc]-PEG. However, the hydrogels with 13% or 13.5% PVA and prepared with PEG200/PEG600 mixture showed higher creep resistance than their annealed counterpart hydrogels prepared with PEG400/PEG600. PVA-PEG hydrogels with PEG400 only showed better creep resistance than their PEG400/PEG600 counterparts. Upon unloading, all gels showed elastic and viscoelastic recovery, which were lower with non-annealed gels than their annealed counterparts (see FIG. 17). Highly porous PVA-[PAAm-co-AAc]-PEG with all formulations showed better recovery than PVA-PEG hydrogels with a higher elastic component.

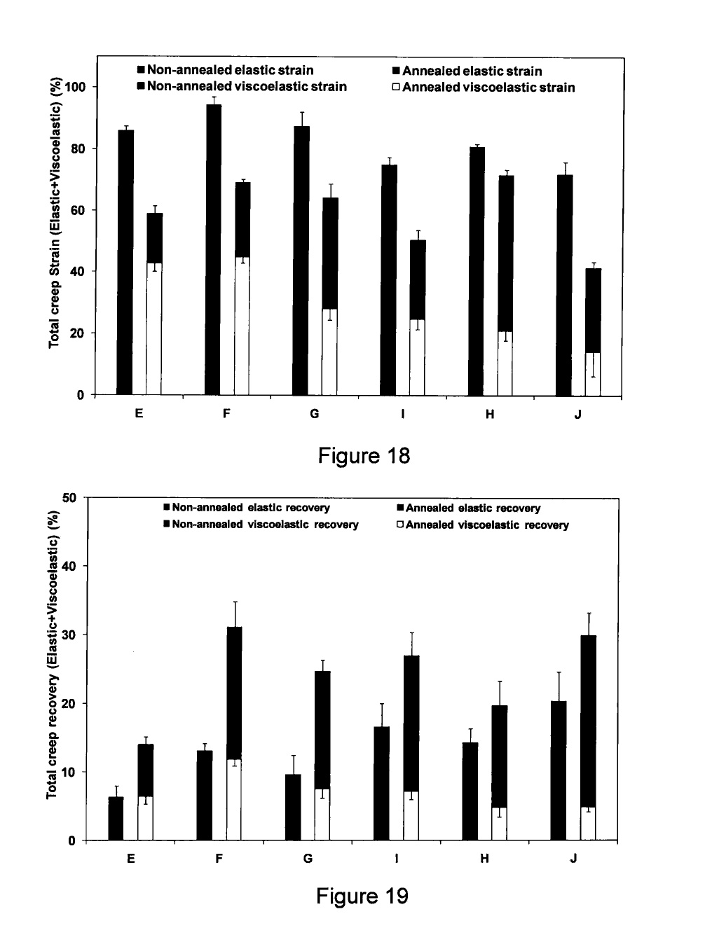

The creep and recovery behavior of the PVA-PEG and PVA-[PAAm]-PEG hydrogels are summarized in FIGS. 18-19 and Table 6 below. Annealing markedly improved the creep resistance of all formulations studied. The creep and recovery behavior of PVA-[PAAm]-PEG hydrogels was similar to those of the highly porous PVA-(PAAm-co-AAc)-PEG hydrogels both before and after annealing (see FIG. 18). PVA-[PAAm]-PEG hydrogels showed slightly lower creep strain than PVA-PEG formulations with the smaller pore structure in non-annealed form. Elastic component of the creep strain Was substantially higher in the PVA-[PAAm]-PEG formulations than PVA-PEG formulation in its annealed form. Annealed gels exhibited higher recovery than their non-annealed counterparts for all formulations. Overall, PVA-[PAAm]-PEG hydrogels recovered more with higher elastic component than the hydrogels prepared with unimodal and bimodal (Example 1) PEG formulations (see FIG. 19).

Table 5 shows total creep strain (TCS), elastic creep strain (ES), viscoelastic creep strain (VS), total creep recovery (TR), elastic creep recovery (ER), viscoelastic creep recovery (VR) of PVA-PEG and PVA-(PAAm-co-AAc)-PEG hydrogels in DP (non-annealed) and AG (annealed) forms. The designations A-J are the same as in Tables 1 and 2.

TABLE 5

| | TCS | ES | VS | TCR | ER | VR |
|---|---|---|---|---|---|---|
| Sample | (%) | (%) | (%) | (%) | (%) | (%) |
| DP-Non-annealed | | | | | | |
| E | 86 ± 1 | 72 ± 1 | 12 ± 2 | 6 ± 2 | 6 ± 2 | 1 ± 0.04 |
| F | 94 ± 3 | 81 ± 3 | 13 ± 2 | 13 ± 1 | 12 ± 2 | 1 ± .13 |
| A | 91 ± 3 | 79 ± 3 | 11 ± 3 | 18 ± 3 | 15 ± 2 | 3 ± .2 |
| C | 73 ± 4 | 63 ± 4 | 10 ± 3 | 19 ± 3 | 14 ± 2 | 5 ± 1 |
| B | 82 ± 3 | 73 ± 3 | 9 ± 2 | 14 ± 2 | 5 ± 1 | 9 ± 2 |
| D | 74 ± 3 | 63 ± 3 | 10 ± 2 | 20 ± 3 | 11 ± 1 | 9 ± 2 |
| AG-Annealed | | | | | | |
| E | 60 ± 3 | 19 ± 3 | 38 ± 2 | 14 ± 1 | 1 ± 0.1 | 13 ± 2 |
| F | 69 ± 1 | 24 ± 11 | 40 ± 2 | 31 ± 3 | 7 ± 0.1 | 24 ± 3 |
| A | 72 ± 3 | 65 ± 4 | 7 ± 2 | 41 ± 3 | 18 ± 4 | 23 ± 3 |
| C | 58 ± 2 | 37 ± 4 | 20 ± 3 | 37 ± 2 | 12 ± 2 | 25 ± 3 |
| B | 72 ± 1 | 66 ± 2 | 6 ± 1 | 28 ± 3 | 16 ± 2 | 12 ± 3 |
| D | 47 ± 1 | 28 ± 3 | 18 ± 2 | 36 ± 2 | 9 ± 2 | 27 ± 2 |

Table 6 below shows total creep strain (TCS), elastic creep strain (ES), viscoelastic creep strain (VS), total creep recovery (TR), elastic creep recovery (ER), viscoelastic creep recovery (VR) of PVA-PEG and PVA-[PAAm]-PEG hydrogels in DP (non-annealed) and AG (annealed) forms. The designations A-J are the same as in Tables 1 and 2.

TABLE 6

| | TCS | ES | VS | TCR | ER | VR |
|---|---|---|---|---|---|---|
| Sample | (%) | (%) | (%) | (%) | (%) | (%) |
| DP-Non-annealed | | | | | | |
| G | 87 ± 5 | 80 ± 7 | 8 ± 2 | 10 ± 3 | 3 ± 1 | 7 ± 2 |
| I | 75 ± 3 | 67 ± 3 | 8 ± 1 | 17 ± 3 | 11 ± 2 | 6 ± 1 |
| H | 81 ± 1 | 74 ± 0.3 | 7 ± 1 | 14 ± 2 | 4 ± 1 | 10 ± 2 |
| J | 72 ± 4 | 61 ± 46 | 11 ± 2 | 20 ± 4 | 12 ± 3 | 1 ± 0.1 |
| AG-Annealed | | | | | | |
| G | 64 ± 5 | 39 ± 7 | 25 ± 3 | 25 ± 2 | 10 ± 2 | 15 ± 3 |
| I | 50 ± 3 | 28 ± 3 | 23 ± 3 | 27 ± 3 | 13 ± 2 | 14 ± 2 |
| H | 72 ± 2 | 53 ± 4 | 19 ± 3 | 20 ± 4 | 10 ± 1 | 10 ± 3 |
| J | 41 ± 2 | 28 ± 7 | 13 ± 7 | 30 ± 3 | 20 ± 2 | 10 ± 1 |

### Example 6

### Tear Strength

The tear strength of PVA-PEG, PVA-[PAAm-co-AAc]-PEG and PVA-[PAAm]-PEG gels was assessed in their DP and AG soak ramp annealing (SRA) rehydrated forms using an MTS Insight 2 mechanical tester. Test samples for tear strength were cut from the molded sheets using a 10 cm long C type die according to ASTM D624. The tear test samples were allowed to equilibrate for 24 hours in DI water at room temperature before testing. The samples were then placed in the wedge grips of the testing machine and deformed under tension until failure. The test was run at a rate of 50 cm/min per ASTM D624.

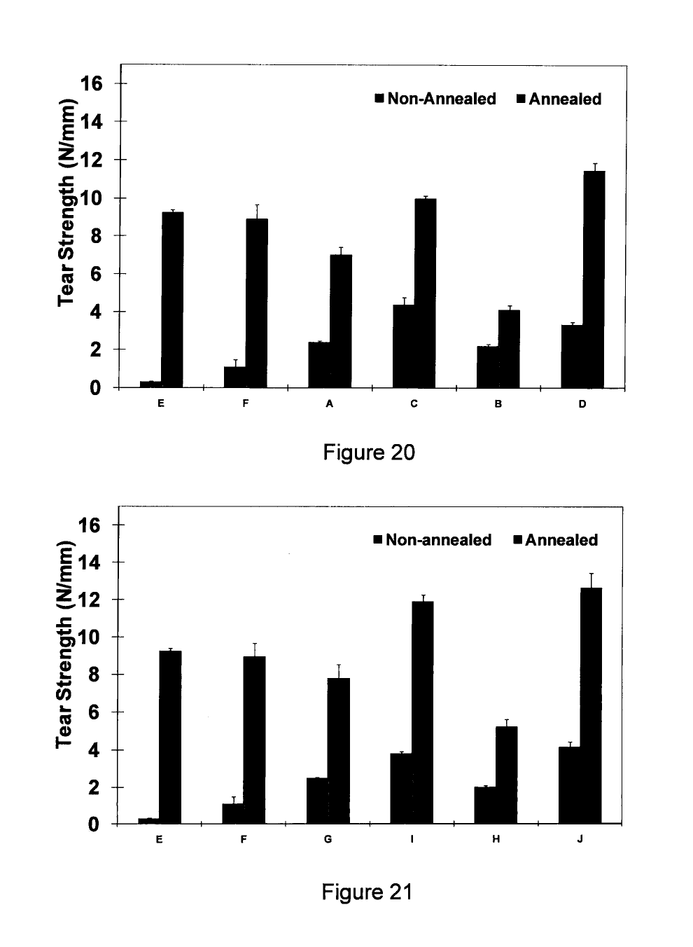

Tear strength of highly porous PVA-[PAAm-co-AAc]-PEG hydrogels were higher than PVA-PEG formulations prepared with unimodal or bimodal PEG in their non-annealed forms (see FIG. 20 and Table 3). In the annealed form of PVA-[PAAm-co-AAc]-PEG with 8.5% or 9% PVA and both with PEG400/PEG600 and PEG200-400 gellant formulations showed lower tear strength than PVA-PEG only hydrogels. PVA-(PAAm-co-AAc)-PEG with 18% PVA showed equal (with PEG400/PEG600) or better (with PEG200/PEG400) tear strength than PVA-PEG mixtures (see FIG. 20).

Highly porous PVA-[PAAm]-PEG hydrogels showed behavior similar to their PVA-(PAAm-co-AAc)-PEG counterparts in terms of tear strength; they exhibited better tear strength than unimodal and bimodal PVA-PEG formulations in their non-annealed form with all formulations (see FIG. 20 and Table 3). PVA-[PAAm]-PEG formulations with 8.5% or 9% PVA showed lower tear strength than PVA-PEG hydrogels in their annealed form. PVA-[PAAm]-PEG mixtures with 13 or 13.5% PVA showed the highest tear strength of all the samples studied (see FIG. 21 and Table 4)

### Example 7

### Relative Coefficient Of Friction (RCOF)

Relative coefficient of friction (RCOF) of PVA-PEG, PVA-[PAAm-co-AAc]-PEG and PVA-[PAAm]-PEG gels was determined in their DP and AG SRA rehydrated forms in DI water at 40° C. while rubbing against an implant-quality finish cobalt-chromium (Co—Cr) surface using a custom annular fixture mounted on a controlled stress rheometer (AR-2000, TA Instruments Inc.) with an inner radius of 1.44 cm and a contact area of 1.42 cm² at a constant angular velocity of 0.1 rad/s. The surface roughness (Rₐ) of the Co—Cr annular fixture was (Rₐ=0.02 μm). The RCOF between the hydrogel and the counterface was calculated using the method of Kavehpour and McKinley (see Kavehpour et al., Tribology Letters, 2004, 17(2): 327-335.4), and Bodugoz-Senturk et al., Biomaterials, 2008, 29(2)141-9, and Oral et a, 55^{rd} Annual Meeting of the Orthopaedic Research Society, Feb. 22-25, 2009, Las Vegas, Nev., USA).

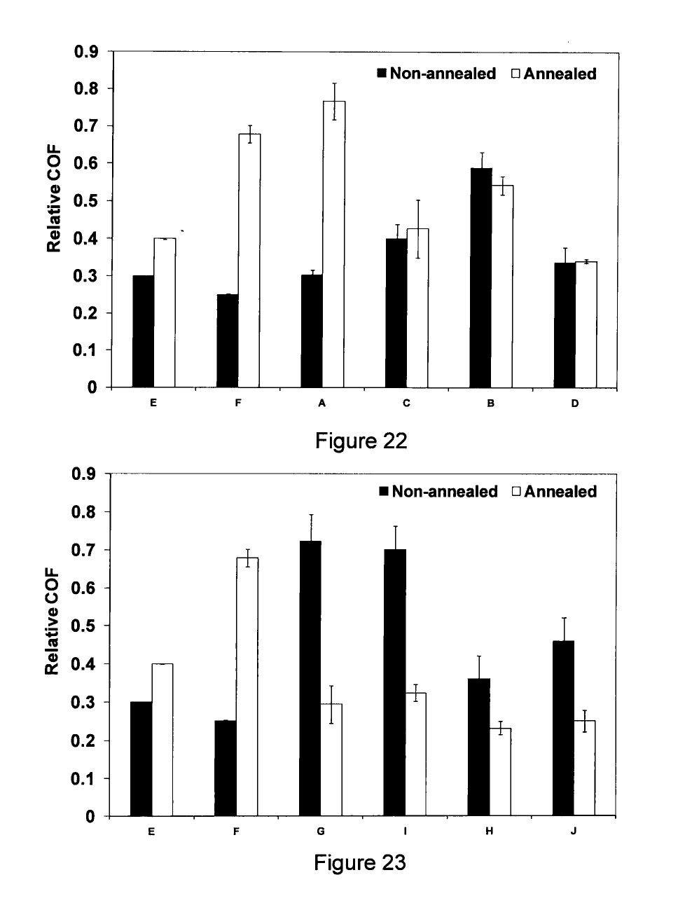

RCOF of highly porous A (Table 1) and C (Table 1) hydrogels was similar to unimodal and bimodal PVA-PEG formulations in their non-annealed forms (see FIG. 22 and Table 3). PVA-(PAAm-co-AAc)-PEG hydrogels with mixtures showed higher RCOF than unimodal and bimodal PVA-PEG hydrogels. Annealing increased the RCOF values of all types of hydrogels with the exception of B (see FIG. 22 and Table 3). In contrast, highly porous PVA-[PAAm]-PEG hydrogels showed smaller RCOF values in their annealed form compared to their non-annealed form (see FIG. 23). RCOF values of PVA-(PAAm-co-AAc)-PEG formulation in the non-annealed form were higher than unimodal and bimodal PVA-PEG formulations (see FIG. 23).

### Example 8

### Effect of Cooling Rate on the Porosity of PVA-(PAAm-Co-AAc)-PEG And Polyethylene-Co-Vinyl Alcohol Hydrogels

We prepared PVA-PAAm-co-AAc-PEG theta gels using two different methods: Method 1: PVA-PAAm-co-AAc-PEG theta gels were prepared by dissolving PVA and PAAm-co-AAc with a mixture of PEG in DI water (Example 1). The final mixture was gelled for 24 hours by cooling down to room temperature. Method 2: PVA-PAAm-co-AAc-PEG theta gels were prepared by dissolving PVA and PAAm-co-AAc with a mixture of PEG in DI water (Example 1). The final mixture was gelled by cooling down to room temperature from 90° C. in a convection oven for 20 hours and kept at room temperature for 24 hours. Both methods resulted in similar morphological structure when cast in sheet form. They were both highly porous composed of 20-200 μm pores connected with channels that were distributed evenly throughout the gel. However, when cast in rod form Method 1 resulted in a gradient structure with finer pores in the outer surface and the larger pores in the core. We did not observe any difference between the rod and sheet for the gels cast with Method 2.

### Example 9

### Preparation of Porous PVA-(PAAm-co-AAc)-PEG and Polyethylene-co-vinyl alcohol (EVAL) Hybrid Hydrogels

Most of the current implant models for cartilaginous tissue repair use sutures, screws, or adhesives (such as fibrin glue) to fix the implants, that is to ensure the implants stay in place. Such methods usually result in damage to the surrounding tissues, or failure of the adhesive before the implant could be secured in the defect. We aimed to prepare a gradient implant which comprises a high strength porous component in the bottom layer and a softer porous component on the top layer. The higher strength component was intended to mimic the bone and the softer component was intended to mimic the cartilage layer. While the top layer of such an implant was designed to enhance the cell growth for cartilage formation, the bottom matrix was designed to serve as a base for bone integration as well as to activate the nutrient flow from the blood stream.

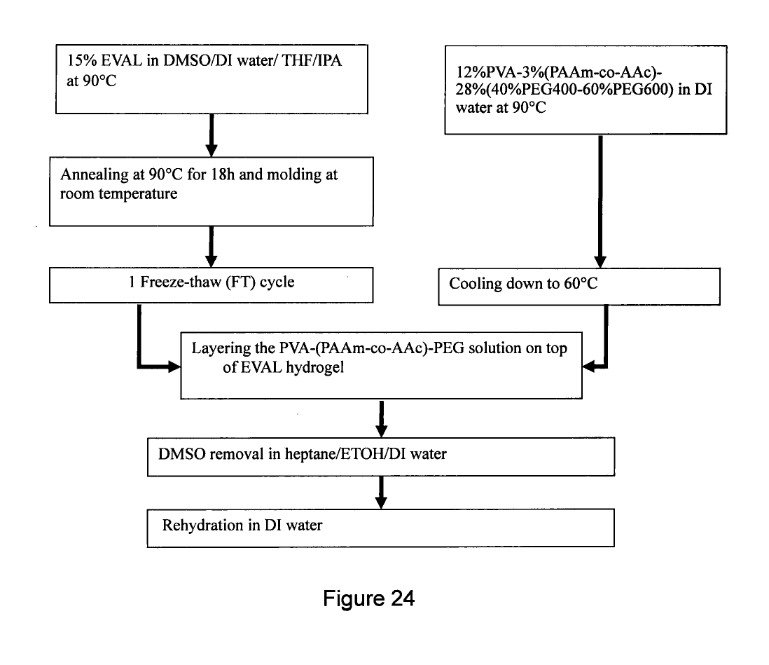

A 15% polyethylene-co vinyl alcohol (EVAL) (w EVAL/w solution mixture×100) was dissolved in mixture of solvent composed of dimethyl sulfoxide (DMSO)/DI water/tetrahydrofuran (THF)/iso-propyl alcohol (60%/20%/15%/5% the values are calculated as [w solvent/w total solvent mixture]×100) (Table 7). The EVAL solution was kept in 90° C. oven for 18 hours then molded between two glass sheets in a custom-made aluminum case. The mold was then frozen for 4 hours, and subsequently thawed for 2 hours. These steps resulted in the gelation of the EVAL. PVA-PAAm-co-AAc-PEG theta gels were prepared by dissolving PVA and PAAm-co-AAc (9% PVA-2% PAAm-co-AAc at 90° C. to a mixture of low and high molecular weight PEG mixture (10% PEG400-15% PEG600) (see Example 1)). The PVA-PAAm-co-AAc-PEG solution was cooled down to 60° C. and molded on top of the EVAL gel (see FIG. 24). The final mixture was first gelled for 24 hours by cooling down to room temperature, then immersed in DI water in order to remove free PEG and unreacted PAAm-co-AAc from the hydrogel on a rotary shaker. The gel was then immersed in n-heptane/ethanol (ETOH)/DI water (10%/10%80%) mixture to remove remaining DMSO and THF. Finally, the resulting gel was immersed in 100% DI water until equilibrium. Equilibrium was determined by periodically weighing the gels.

Table 7 below shows PVA-(PAAm-co-AAc)-PEG and Polyethylene-co-vinyl alcohol (EVAL) hybrid hydrogels formulations.

TABLE 7

| | | Solvent mixture for EVAL | | | | | PAAm- | | |
|---|---|---|---|---|---|---|---|---|---|
| | EVAL | hydrogel (%) | | | | PVA | co-AAc | PEG (%) | |
| Sample | (%) | DMSO | DI water | THF | IPA | (%) | (%) | 400 | 600 |
| EVAL/PVA- | 15 | 60 | 20 | 15 | 5 | 9 | 2 | 10 | 15 |
| (PAAm-co-AAc)- | | | | | | | | | |
| PEG400/600 | | | | | | | | | |

Thus, the invention provides methods of making and using fabricated creep resistant, highly lubricious, tough hydrogels including polyvinyl alcohol-polyacrylamide-co-acrylic acid copolymer hydrogels, and creep resistant, highly lubricious, tough, and ionic hydrogel-containing compositions for cartilage repair or as interpositional devices.

Each reference disclosed in the present application is incorporated by reference herein in its entirety.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention.

1. A method of making a creep resistant, highly lubricious, tough hydrogel, the method comprising:
(a) preparing a first solution including a first polymer and polyacrylamide-co-acrylic acid; and
(b) introducing a second solution including a gellant into the first solution to form the hydrogel.

2. The method of claim 1 wherein:
after introducing the second solution into the first solution, a combination of the first solution and the second solution has a Flory interaction parameter that is sufficient for gelation.

3. The method of claim 1 wherein:
step (a) further comprises heating the first solution to a first temperature above room temperature, and
step (b) further comprises cooling a combination of the first solution and the second solution to a second temperature at or below theta temperature.

4. (canceled)

5. (canceled)

6. (canceled)

7. (canceled)

8. The method of claim 1 wherein:
the gellant is polyethylene glycol, and
the first polymer is poly(vinyl alcohol).

9. The method of claim 8 wherein:
the polyethylene glycol has a molecular weight distribution with more than one mode.

10. (canceled)

11. (canceled)

12. (canceled)

13. (canceled)

14. (canceled)

15. The method of claim 8 wherein:
a total polymer content of poly(vinyl alcohol) and polyacrylamide-co-acrylic acid in a combination of the first solution and the second solution is in the range of 1 wt % to 50 wt %.

16. (canceled)

17. (canceled)

18. (canceled)

19. (canceled)

20. (canceled)

21. (canceled)

22. The method of claim 1 wherein:
step (b) further comprises providing a mold containing a second hydrogel, and placing a combination of the first solution and the second solution into the mold to contact the second hydrogel thereby forming a hybrid hydrogel including the hydrogel and the second hydrogel.

23. (canceled)

24. (canceled)

25. (canceled)

26. The method of claim 1 wherein:
the hydrogel includes channels of interconnected pores, and
the channels have an average diameter in cross-section between 2 and 100 micrometers.

27. (canceled)

28. (canceled)

29. (canceled)

30. (canceled)

31. (canceled)

32. A method of making a creep resistant, highly lubricious, tough hydrogel, the method comprising:
(a) preparing an aqueous mixture including a first polymer and polyacrylamide-co-acrylic acid; and
(b) subjecting the mixture to one or more freeze-thaw cycles to form the hydrogel.

33. The method of claim 32 wherein:
the first polymer is polyvinyl alcohol).

34. The method of claim 1 or claim 32 further comprising:
(c) annealing the hydrogel at a temperature below the melting point of the hydrogel.

35. The method of claim 1 or claim 32 further comprising:
(c) dehydrating the hydrogel under an inert environment or in a dehydrating solvent.

36. (canceled)

37. The method of claim 32 further comprising:
(c) dehydrating the hydrogel; and
(d) annealing the hydrogel at a temperature of about 80° C. to about 200° C.

38. The method of claim 32 further comprising:
(c) dehydrating the hydrogel; and
(d) rehydrating the hydrogel by soaking in a saline solution or in water.

39. The method of claim 32 further comprising:
(c) contacting the hydrogel with an organic solvent, wherein the hydrogel is not soluble in the solvent, and wherein the solvent is at least partially miscible in water;
(d) heating the hydrogel to a temperature below or above the melting point of the hydrogel; and
(e) cooling the heated hydrogel to room temperature.

40. (canceled)

41. (canceled)

42. The method of claim 32 further comprising:
(c) dehydrating the hydrogel by placing the hydrogel in (i) a non-solvent selected from the group consisting of polyethylene glycol, alcohols, acetones, saturated salinated water, vitamin, carboxylic acids, and aqueous solutions of a salt of an alkali metal, or (ii) in a supercritical fluid.

43. (canceled)

44. (canceled)

45. (canceled)

46. (canceled)

47. A hydrogel made by a method according to claim 1.

48. (canceled)

49. A medical implant comprising a hydrogel made by a method according to claim 1.

50. (canceled)

51. (canceled)

52. (canceled)

53. (canceled)

54. (canceled)

55. (canceled)

56. (canceled)

57. A medical implant comprising:
a first layer comprising a first hydrogel made by a method according to claim 1; and
a second layer attached to the first layer, the second layer comprising a second material selected from metallic materials, ceramic materials and polymeric materials.

58. The medical implant of claim 57 wherein:
the second layer comprises a second hydrogel made by a method according to claim 1, and
the first hydrogel and the second hydrogel have different properties.

59. The medical implant of claim 58 wherein:
the first hydrogel and the second hydrogel have different pore structures.

60. (canceled)

61. (canceled)

62. (canceled)

63. (canceled)

64. (canceled)

65. (canceled)
